(19) United States
(12) Patent Application Publication
(10) Pub. No.: US 2025/0191736 A1
(43) Pub. Date: Jun. 12, 2025

(54) WORKFLOWS AND GRAPHICAL USER INTERFACES FOR OPTICAL VIEWING DEVICES

(71) Applicant: Welch Allyn, Inc., Skaneateles Falls, NY (US)

(72) Inventors: David Villarreal, Fayetteville, NY (US); Danielle R. Endres, Liverpool, NY (US); Lei Guo, Manlius, NY (US); David G. Perkins, Tully, NY (US)

(21) Appl. No.: 18/951,999

(22) Filed: Nov. 19, 2024

Related U.S. Application Data
(60) Provisional application No. 63/607,600, filed on Dec. 8, 2023.
(60) Provisional application No. 63/617,234, filed on Jan. 3, 2024.

(51) Int. Cl. G16H 30/40; A61B 1/04; A61B 1/227; A61B 3/13

(52) U.S. Cl. CPC G16H 30/40; A61B 1/04; A61B 1/227; A61B 3/13

(57) ABSTRACT

A device for imaging an anatomy includes a bracket for attaching the device to an instrument head of an optical viewing device, the instrument head including an eyepiece for examining the anatomy. The device includes a camera that captures images through the eyepiece of the instrument head, and a display screen that displays the images. The device determines a type of the optical viewing device, and presents a workflow on the display screen based on the type of the optical viewing device. The workflow enables capture of a video of the anatomy viewed through the eyepiece of the instrument head. The workflow includes one or more tools for annotating one or more frames selected from the video.

### CROSS REFERENCE TO RELATED APPLICATIONS

This application claims priority to U.S. Provisional Patent Application No. 63/607,600, filed Dec. 8, 2023, and U.S. Provisional Patent Application No. 63/617,234, filed Jan. 3, 2024, the disclosures of which are hereby incorporated by reference in their entireties.

### BACKGROUND

Optical viewing devices are used for examining patients as part of routine examinations. Examples of optical viewing devices can include, without limitation, an otoscope for assessing the ears of a patient, an ophthalmoscope for assessing the eyes of a patient, and a dermatoscope for assessing the skin of a patient. Different types of examinations and workflows are typically performed based on the type of optical viewing device being used.

### SUMMARY

In general terms, the present disclosure relates to imaging for optical viewing devices. In one possible configuration, an imaging device recognizes a type of optical viewing device, and presents a workflow based on the type of optical viewing device. Various aspects are described in this disclosure, which include, but are not limited to, the following aspects.

One aspect relates to a device for imaging an anatomy, the device comprising: a bracket for attaching the device to an instrument head of an optical viewing device, the instrument head including an eyepiece for examining the anatomy; a camera that captures images through the eyepiece of the instrument head; a display screen that displays the images captured by the camera; at least one processing device; and at least one computer readable data storage device storing software instructions that, when executed by the at least one processing device, cause the device to: determine a type of the optical viewing device; and present a workflow on the display screen based on the type of the optical viewing device, the workflow enabling capture of a video of the anatomy viewed through the eyepiece of the instrument head, and the workflow including one or more tools for annotating one or more frames selected from the video.

Another aspect relates to a method of imaging an anatomy, the method comprising: determining a type of an optical viewing device, the type of the optical viewing device selected from the group consisting of an ophthalmoscope, an otoscope, and a dermatoscope; and presenting a workflow on the imaging device, the workflow being presented based on the type of the optical viewing device, the workflow enabling capture of a video of the anatomy viewed through an eyepiece of the instrument head, and the workflow including one or more tools for annotating one or more frames selected from the video.

Another aspect relates to a system for imaging an anatomy, the system comprising: an optical viewing device including an instrument head having an eyepiece for examining the anatomy; and an imaging device configured for attachment to the optical viewing device, the imaging device including: a camera for capturing images through the eyepiece of the instrument head; a display screen for displaying the images captured by the camera; at least one processing device communicatively connected to the camera and the display screen; and at least one computer readable data storage device storing software instructions that, when executed by the at least one processing device, cause the imaging device to: determine a type of the optical viewing device; and present a workflow on the display screen based on the type of the optical viewing device, the workflow enabling capture of a video of the anatomy viewed through the eyepiece of the instrument head, and the workflow including one or more tools for annotating one or more frames selected from the video.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 shows examples of different types of optical viewing devices, each optical viewing device is shown attached to an imaging device.

FIG. 2 is an isometric view of an example of an optical viewing device shown in FIG. 1, the optical viewing device shown from a physician perspective.

FIG. 3 is another isometric view of the optical viewing device of FIG. 2, the optical viewing device shown from a patient perspective.

FIG. 4 is an isometric view of an example of the imaging device attached to the optical viewing device of FIG. 2, the imaging device shown from the physician perspective.

FIG. 5 is a front isometric view of the imaging device of FIG. 4.

FIG. 6A is a front view of the imaging device of FIG. 4.

FIG. 6B is a rear view of the imaging device of FIG. 4.

FIG. 6C is a top view of the imaging device of FIG. 4.

FIG. 7 is an isometric view showing a camera of the imaging device of FIG. 4.

FIG. 8 is an isometric view of another example of the imaging device attached to the optical viewing device of FIG. 2, the imaging device shown from the physician perspective.

FIG. 9 is an isometric view of the imaging device of FIG. 8 before attachment to the optical viewing device of FIG. 2, the imaging device shown from the patient perspective.

FIG. 10 is an isometric view of a charging station for charging the optical viewing devices and the imaging device of FIG. 1.

FIG. 11 schematically illustrates an example of a method of optimizing a workflow presented on the imaging device of FIGS. 1-10.

FIG. 12 illustrates an example of a workflow that can be provided on the imaging device of FIGS. 1-10 in accordance with the method of FIG. 11.

FIGS. 13-26 illustrate examples of graphical user interfaces that can be displayed on the imaging device of FIGS. 1-10 when attached to a type of optical viewing device, the graphical user interfaces presented in accordance with the workflow of FIG. 12.

FIGS. 27-36 illustrate examples of graphical user interfaces that can be displayed on the imaging device of FIGS. 1-10 when attached to another type of optical viewing device, the graphical user interfaces presented in accordance with the workflow of FIG. 12.

FIGS. 37-49 illustrate examples of graphical user interfaces that can be displayed on the imaging device of FIGS. 1-10 when attached to another type of optical viewing device, the graphical user interfaces presented in accordance with the workflow of FIG. 12.

FIG. 50 illustrates an exemplary architecture of a computing device of the imaging device shown in any of the above figures.

### DETAILED DESCRIPTION

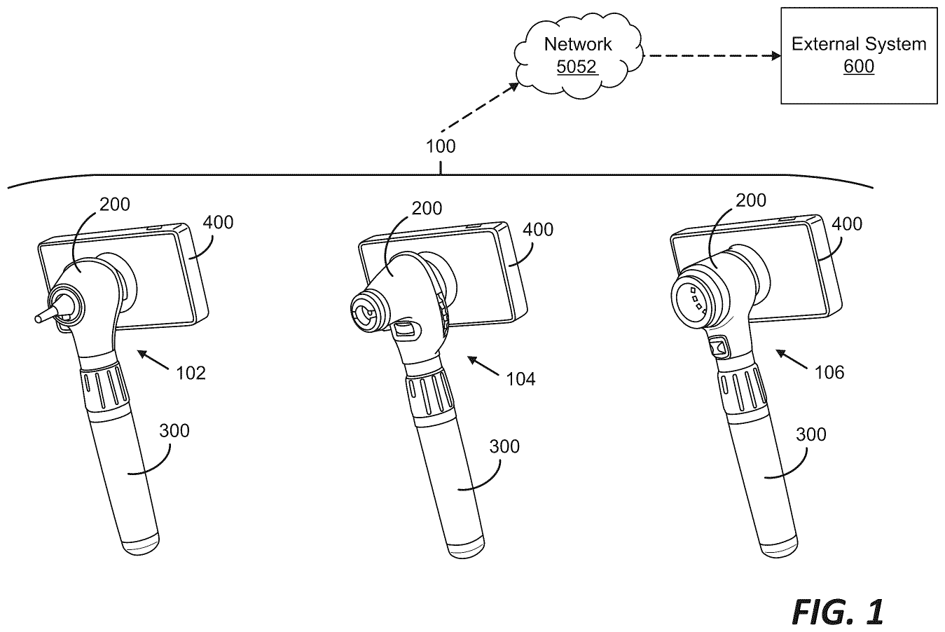

FIG. 1 shows examples of different types of optical viewing devices 100. For example, the optical viewing devices 100 include a first type of optical viewing device 102 such as an otoscope, a second type of optical viewing device 104 such as an ophthalmoscope, and a third type of optical viewing device 106 such as a dermatoscope. Additional types of the optical viewing devices 100 are possible, and the disclosure provided herein is not limited to otoscopes, ophthalmoscopes, and dermatoscopes.

As shown in FIG. 1, each type of optical viewing device 100 includes an instrument head 200 attached to an instrument handle 300. The instrument head 200 can include a light source and optics for viewing an anatomical area of interest through an eyepiece. The instrument handle 300 can include a power source that powers the light source and other components of the instrument head 200. For example, the instrument handle 300 can include rechargeable batteries, disposable batteries, or a tether to a wall transformer for supplying electrical power to the components of the instrument head 200.

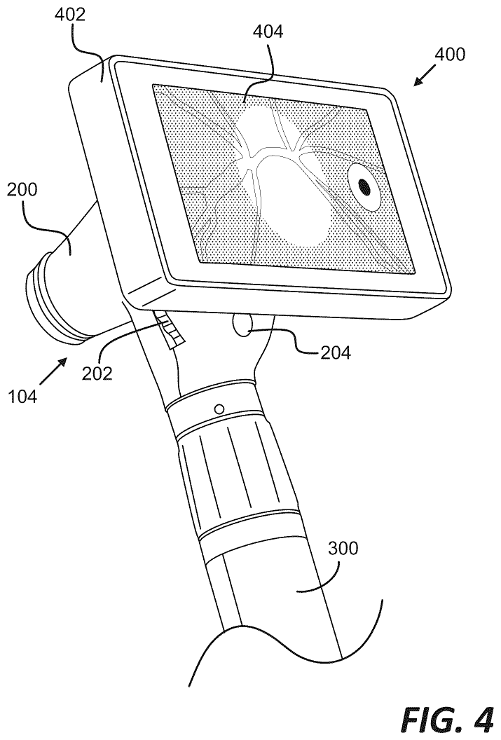

As further shown in FIG. 1, an imaging device 400 is attached to the instrument head 200 of each type of optical viewing device 100. The imaging device 400 is a portable, battery powered camera that can record high quality image frames and videos from the optical viewing devices 100, providing digital imaging solutions. For example, the imaging device 400 captures images through an eyepiece of the instrument head 200 for display on a display screen 404 (see FIG. 4) for viewing by a physician. The images captured by the imaging device 400 can be analyzed by algorithms (including artificial intelligence algorithms) for disease screening, and the images can be stored in an electronic medical record (EMR) of a patient.

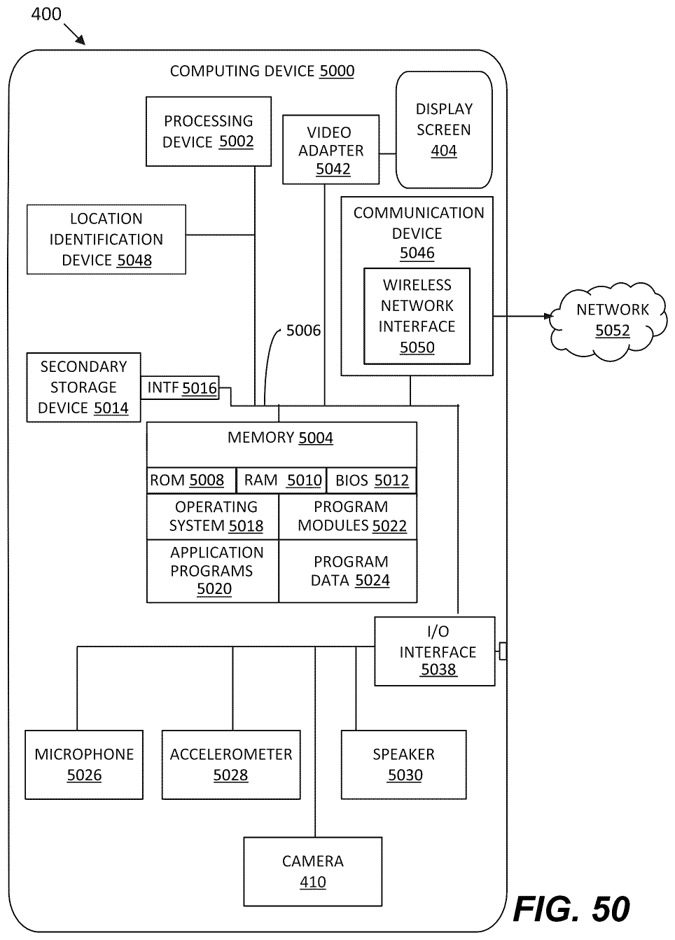

In some examples, the imaging device 400 transmits images, videos, and other data to an external system 600, which analyzes the images, videos, and other data to generate one or more results for transmission back to the imaging device 400. The external system 600 can be remotely located with respect to the optical viewing device 100 and the imaging device 400. In some examples, the external system 600 includes a cloud server. The imaging device 400 can communicate with the external system 600 via a network 5052 (see also FIG. 50).

The algorithms (including artificial intelligence algorithms) for disease screening can be executed on either or both of the imaging device 400 and the external system 600. In some examples, the external system 600 may also host storage of the images, videos, and other data received from the imaging device 400. In further examples, the external system 600 can host the EMR of the patient. In yet further examples, the external system 600 may provide connectivity to other external systems and servers having image storage, or that host the EMR.

As shown in FIG. 1, the imaging device 400 is universal with respect to the first type of optical viewing device 102, the second type of optical viewing device 104, and the third type of optical viewing device 106 such that the imaging device 400 can be interchangeably used between the different types of optical viewing devices. The instrument handle 300 may also be universal with respect to the first type of optical viewing device 102, the second type of optical viewing device 104, and the third type of optical viewing device 106 such that the instrument handle 300 can be interchangeably used between the different types of optical viewing devices.

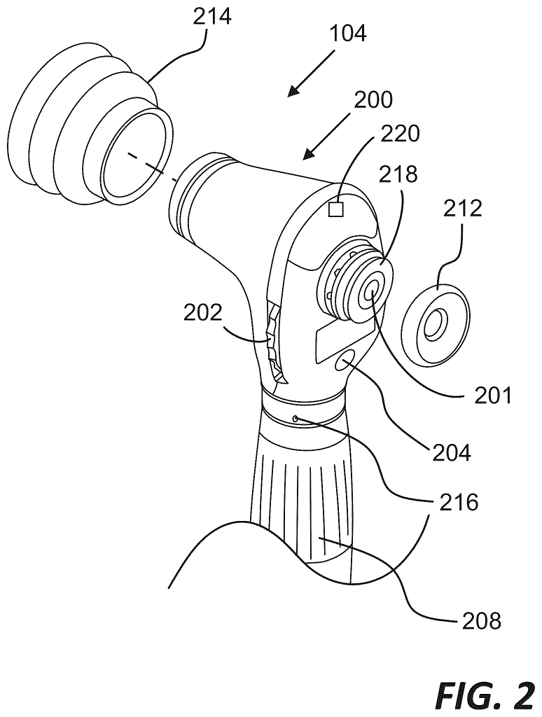

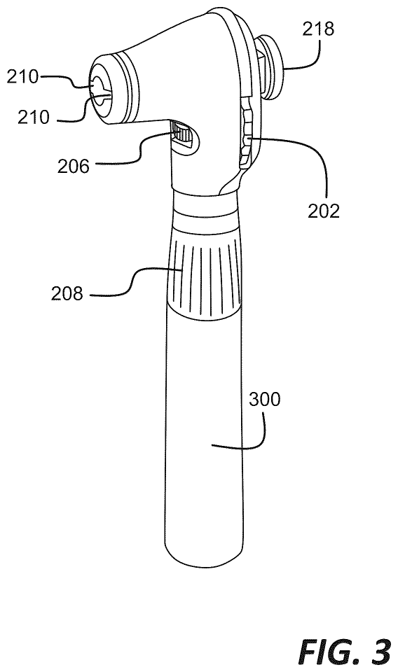

FIGS. 2 and 3 are isometric views of an example of the second type of optical viewing device 104. In FIG. 2, the second type of optical viewing device 104 is shown from a physician perspective. In FIG. 3, the second type of optical viewing device 104 is shown from a patient perspective. As discussed above, the second type of optical viewing device 104 is an ophthalmoscope. While FIGS. 2 and 3 refer to the second type of optical viewing device 104, the first type of optical viewing device 102 and the third type of optical viewing device 106 can include similar components and features such that the following description similarly applies to the first type of optical viewing device 102 and the third type of optical viewing device 106.

Referring now to FIGS. 2 and 3, the second type of optical viewing device 104 includes a diopter focus wheel 202 and a diopter readout 204. The diopter focus wheel 202 can be used to adjust a focus of an eyepiece 201. The diopter focus wheel 202 can be used to correct the refractive errors of the user of the optical viewing device 104 and the patient. For example, the diopter focus wheel 202 can be used to provide a positive dioptric value to accommodate for hyperopia eyesight (farsightedness) of both the user of the optical viewing device 104 and the patient, and to provide a negative dioptric value to accommodate for myopia eyesight (nearsightedness) of both the user of the optical viewing device 104 and the patient. The dioptric values adjusted by using the diopter focus wheel 202 are displayed in the diopter readout 204.

The second type of optical viewing device 104 can further include a filter wheel 206 to select a filter for viewing through the eyepiece 201. For example, the filter wheel 206 can be used to select a reticle target to measure the optic disc, a cobalt blue filter to detect corneal abrasions, a red-free filter, and additional types of filters.

The second type of optical viewing device 104 can further include a light control 208 for controlling illumination from the light source, disc alignment lights 210 (e.g., red for right eye exams; yellow for left eye exams), an eyepiece bumper 212, an optional patient eye cup 214, an optional locking collar 216, and an eyepiece housing 218. As will be described in more detail below, the imaging device 400 includes a bracket that removably attaches to the eyepiece housing 218 for securing the imaging device 400 to the instrument head 200.

As further shown in FIG. 2, the second type of optical viewing device 104 can include an identifier 220. While the identifier 220 is described with reference to the second type of optical viewing device 104, the first and third types of optical viewing devices 102, 106, as well as additional types of optical viewing devices can similarly include the identifier 220, as described herein. As will be described in more detail, the identifier 220 provides machine-readable data that can be detected by the imaging device 400 to detect attachment of the imaging device 400 to the instrument head 200, and to convey additional information such as the type of the instrument head 200 (i.e., whether the instrument head 200 is for an otoscope, ophthalmoscope, dermatoscope, or other type of optical viewing device).

In some examples, the identifier 220 is a wireless antenna that transmits a wireless signal that is detected by the imaging device 400 when the imaging device 400 is attached to the instrument head 200. In some examples, the wireless signal transmitted by the identifier 220 to the imaging device 400 can include a radio frequency identification (RFID) signal, a near field communication (NFC) signal, a Bluetooth signal, a Wi-Fi signal, or other similar wireless signals. The identifier 220 can include a passive antenna or tag that is activated by an active antenna on the imaging device 400 to transmit the wireless signal when the imaging device 400 is in close proximity to the instrument head 200 such as when it is attached thereto.

In some further examples, the identifier 220 can provide additional types of machine-readable data that can be detected by the imaging device 400. For example, the identifier 220 can include a quick response (QR) code or other type of machine-readable label that can be read by a primary camera or a secondary camera of the imaging device 400.

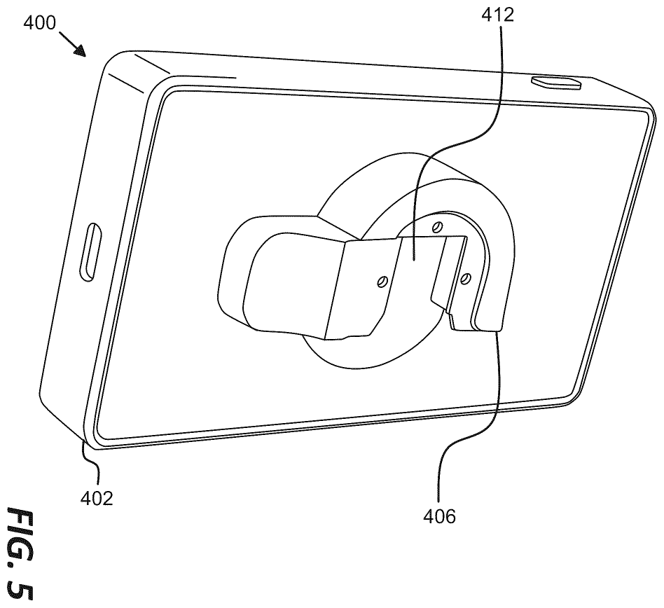

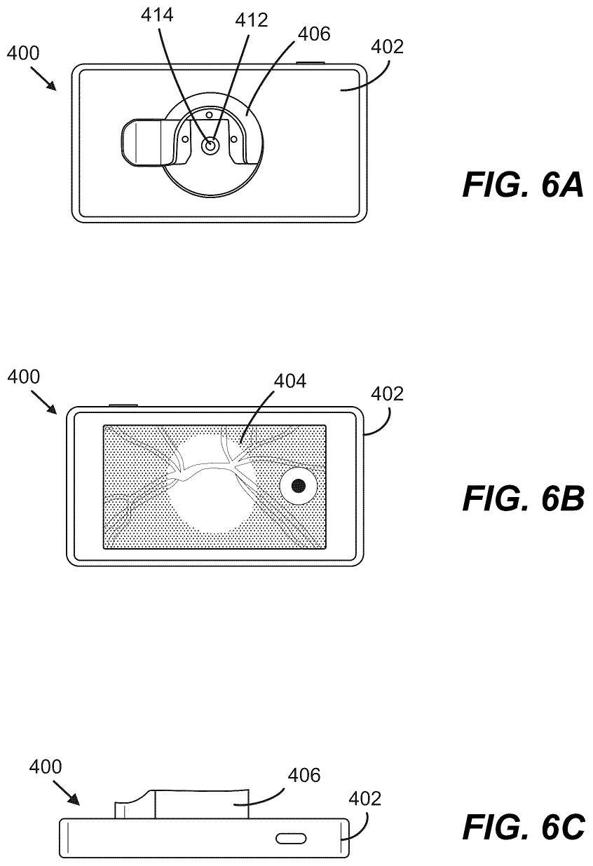

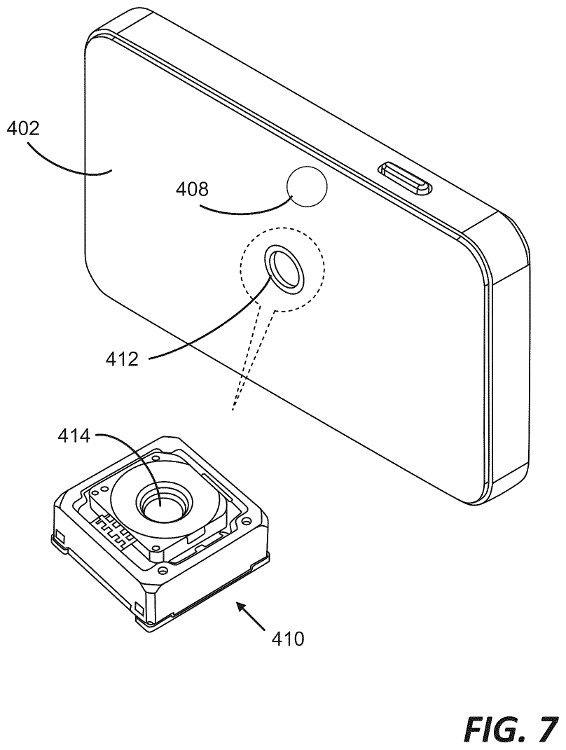

FIG. 4 is an isometric view of an example of the imaging device 400 attached to the second type of optical viewing device 104. In FIG. 4, the imaging device 400 shown from the physician perspective. FIG. 5 is a front isometric view of the imaging device 400. FIG. 6A is a front view of the imaging device 400. FIG. 6B is a rear view of the imaging device 400. FIG. 6C is a top view of the imaging device 400. FIG. 7 is an isometric view showing a camera 410 of the imaging device 400. Referring now to FIGS. 4-7, the imaging device 400 captures images viewed from the eyepiece 201 of an optical viewing device 100.

While FIGS. 4-6 show the imaging device 400 attached to the second type of optical viewing device 104, the imaging device 400 can be similarly attached to the eyepiece housings of the first type of optical viewing device 102 and the third type of optical viewing device 106 for capturing images viewed from the eyepieces of the first type of optical viewing device 102 and the third type of optical viewing device 106.

As shown in FIGS. 4-6, the imaging device 400 includes a housing 402. In this example, a bracket 406 is integrated with a back surface of the housing 402. The bracket 406 allows the imaging device 400 to physically attach to the optical viewing devices 100. For example, the bracket 406 can be fixed around an eyepiece housing 218 (see FIGS. 2 and 3) for attaching the imaging device 400 to an instrument head 200. In alternative examples, the bracket 406 can be part of an accessory case that attaches to the housing 402, and that can be used to physically attach the imaging device 400 to the optical viewing devices 100.

As shown in FIGS. 5 and 7, the housing 402 further includes an aperture 412 for a lens 414 of the camera 410. The camera 410 is mounted inside the housing 402 of the imaging device 400. When the imaging device 400 is mounted to the instrument head 200, the camera 410 is aligned with the eyepiece 201 of the instrument head 200 for capturing images viewed through the eyepiece 201 of the instrument head 200. The camera 410 is centrally mounted inside the housing 402 to provide even balance and weight distribution for when the imaging device 400 is attached to the instrument head 200, thereby improve the ergonomics of the assembly. A protrusion of the lens 414 beyond the back surface of the housing 402 is minimized such that the lens 414 is substantially flush with the back surface of the housing 402.

The camera 410 can include features such as auto focus, auto-exposure, auto white-balance, and image stabilization. The camera 410 can include a 12 MP color image sensor. As an illustrative example, the camera 410 can include an equivalent focal length (on 35 mm film) of 52-77 mm, 4K (30 FPS) video recording with 4000×3000 pixel resolution, and a record time of 90minutes at 4K resolution, 1 minute per clip. Alternative camera parameters are possible.

The housing 402 is compact and lightweight. In some examples, the housing 402 includes a protective overmold having a base layer of plastic material, and a top layer of rubber to provide shock absorption and improved grip. The housing 402 can include one or more ports such as a USB-C port for charging the battery, and for data transferring including uploading images and videos captured by the camera 410 to another device. As an illustrative example, the housing 402 can have a thickness (e.g., distance between the lens 414 of the camera 410 and the display screen 404) that is less than 25 mm, and a weight that is less than 250g. The housing 402 can include a power button to turn on/off and wake up the imaging device 400. The housing 402 houses an integrated, rechargeable battery that can, for example, power 90 minutes of 4K video recording by the camera 410, and 3-4 hours of screen time on the display screen 404.

As shown in FIG. 7, the imaging device 400 can include a detector 408 that detects the machine-readable data from the identifier 220 on the instrument head 200 (see FIG. 2) to detect attachment of the imaging device 400 to the instrument head 200, and to detect additional information such as the type of the instrument head 200 (i.e., whether the instrument head 200 is for an otoscope, ophthalmoscope, dermatoscope, or other type of optical viewing device).

In some examples, the detector 408 is a wireless antenna that detects a wireless signal from the identifier 220 on the instrument head 200 when the imaging device 400 is attached to the instrument head 200. In some examples, the detector 408 on the imaging device 400 can detect a radio frequency identification (RFID) signal, a near field communication (NFC) signal, a Bluetooth signal, a Wi-Fi signal, or other similar wireless signals emitted from the instrument head 200. The detector 408 can include an active antenna or tag that activates a passive antenna or tag on the instrument head 200 to receive a transmission of the wireless signal. In some examples, the active antenna is mounted on the imaging device 400 in a location that corresponds to the placement of the passive antenna on the instrument head 200 such that the active antenna activates the passive antenna when in close proximity to the passive antenna such as when the imaging device 400 is attached to the instrument head 200.

In some further examples, the detector 408 can include a secondary camera that can read a quick response (QR) code or other type of machine-readable label placed on the instrument head 200. The secondary camera can read the machine-readable label to detect attachment of the imaging device 400 to the instrument head 200, as well as to determine the type of the instrument head 200 (i.e., whether the instrument head 200 is for an otoscope, ophthalmoscope, dermatoscope, or other type of optical viewing device). The secondary camera can be mounted on the imaging device 400 in a location that corresponds to the placement of the machine-readable label on the instrument head 200.

As further shown in FIGS. 4-7, the imaging device 400 includes the display screen 404 for displaying the images captured by the camera 410. In some examples, the display screen 404 is a touchscreen such that it can both display the images, and receive inputs from a user. For example, the display screen 404 can be used by a user of the imaging device to: adjust the settings of the camera 410 (e.g., focus, exposure, white balance, FOV/zoom); tapping the display screen 404 to trigger focus and lock; adjust settings of the display screen 404 such as the screen brightness; provide a virtual keyboard to type in information; display a battery-life indicator; provide video recording controls (e.g., start, stop, save, delete, review, upload; provide a sliding bar to go through video frames, pinch-zoom to enlarge; display arrow(s) to indicate image orientation; and display one or more stamps (e.g., date, time, filter info, etc.) on saved images.

The display screen 404 can include a true color multi-touch screen (in-plane switching (IPS), or light-emitting diode (LED)). The display screen 404 can have a bezel-less design (e.g., full-screen display). The display screen 404 can have a resolution of at least 250 pixels per inch (PPI), a diagonal screen size of about 2 inches to about 5 inches, an aspect ratio of 16:9/4:3, a maximum brightness of 500 nits. The display screen 404 can also include features such as screen auto off, and wake up by power button or tapping the display screen 404.

Additionally, the imaging device 400 can provide haptic feedback based on touches detected on the display screen 404, or selection of one or more physical push buttons on the housing 402 of the imaging device 400. The haptic feedback can include vibrations to give feedback rather than audible beeps to quickly communicate to the user actions such as when video capture has initiated or when a captured video is ready for playback.

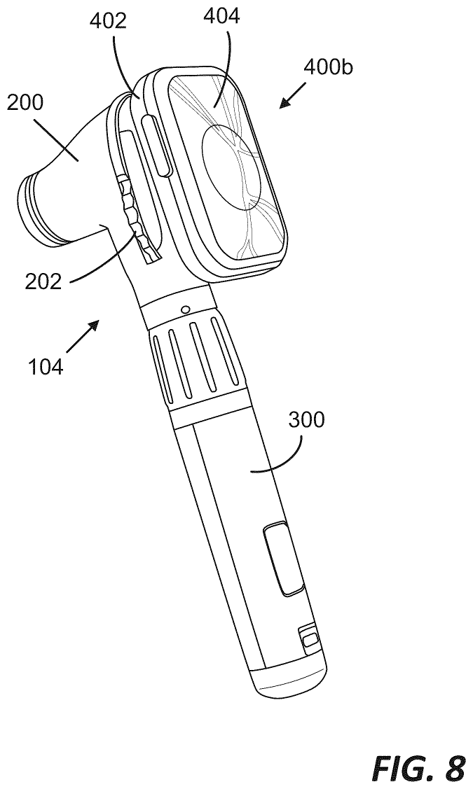

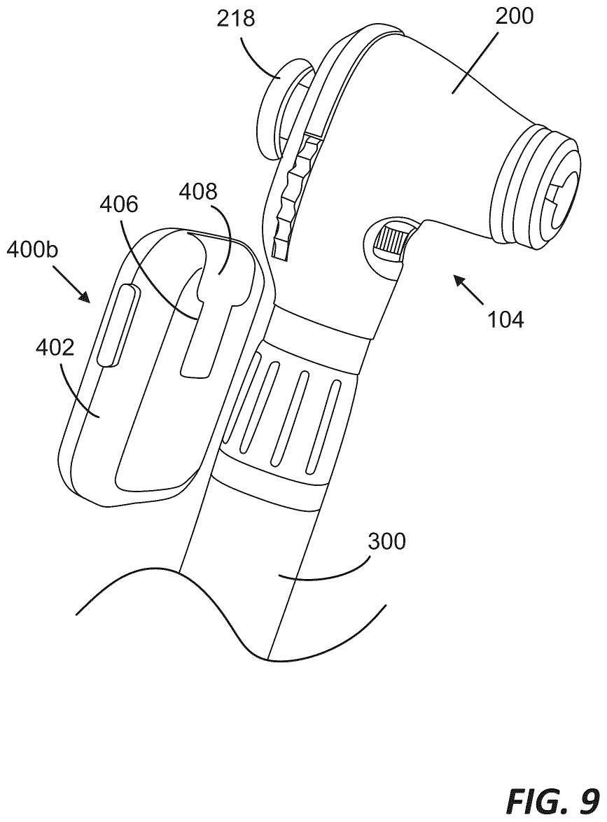

FIG. 8 is an isometric view of another example of an imaging device 400 attached to the second type of optical viewing device 104. In FIG. 8, the imaging device 400 is shown from the physician perspective. FIG. 9 is an isometric view of the imaging device 400 before attachment to the second type of optical viewing device 104. In FIG. 9, the imaging device 400 is shown from the patient perspective. While FIGS. 8 and 9 show the imaging device 400 attached to the second type of optical viewing device 104, the imaging device 400 can similarly attach to the first type of optical viewing device 102, to the third type of optical viewing device 106, and to additional types of optical viewing devices for capturing and displaying images captured from the eye pieces of the first type of optical viewing device 102, the third type of optical viewing device 106, and the additional types of optical viewing devices.

The imaging device 400 is similar to the imaging device 400 shown in FIGS. 4-7. For example, the imaging device 400 includes a housing 402 having a bracket 406 for attaching to the eyepiece housing 218 of the optical viewing device 100. The imaging device 400 similarly includes a display screen 404 for displaying images captured by a camera that is centrally mounted inside the housing 402 of the imaging device 400 to provide even balance and weight distribution. Like in the examples described in FIGS. 4-7, the camera of the imaging device 400 is configured for alignment with the eyepiece 201 of the instrument head 200 for capturing images viewed through the eyepiece 201 of the instrument head 200.

The imaging device 400 can also include a detector 408 for detecting machine-readable data from the instrument head 200 such as to detect attachment of the imaging device 400 to the instrument head 200, and to detect additional information from the instrument head 200 such as the type of the instrument head 200 (i.e., whether the instrument head 200 is for an otoscope, ophthalmoscope, dermatoscope, or other type of optical viewing device).

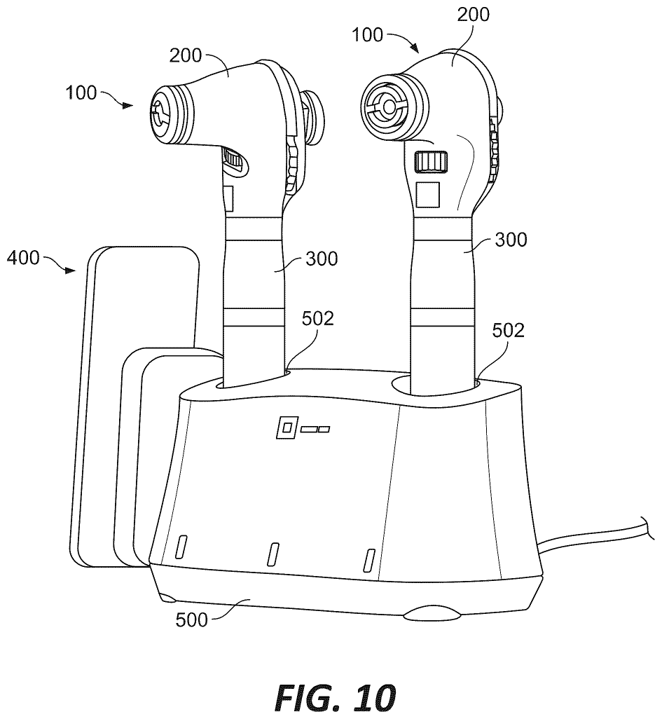

FIG. 10 is an isometric view of a charging station 500 for charging the optical viewing devices 100. For example, each instrument handle 300 can be inserted into an aperture 502 of the charging station 500 for charging the power source in the instrument handle 300 when the optical viewing device 100 is not being used. As further shown in FIG. 10, the imaging device 400 can also be held on the charging station 500 for storage and charging.

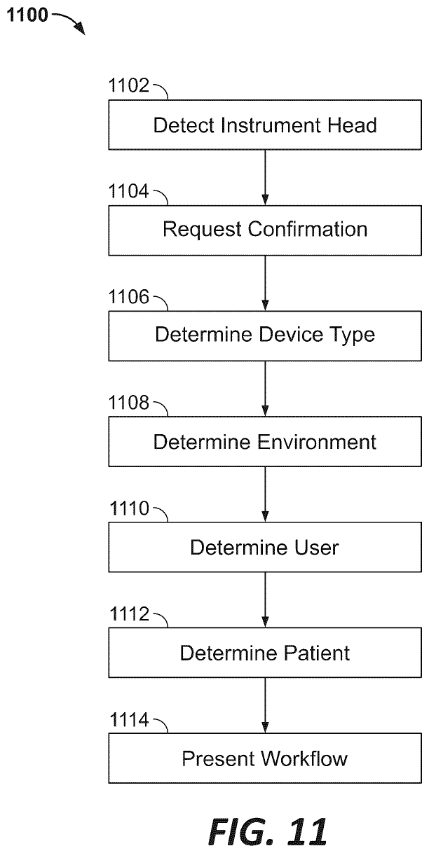

FIG. 11 schematically illustrates an example of a method 1100 of optimizing a workflow presented on the imaging device 400. The method 1100 improves the universality and usability of the imaging device 400 with respect to the first type of optical viewing device 102 (e.g., otoscope), the second type of optical viewing device 104 (e.g., ophthalmoscope), the third type of optical viewing device 106 (e.g., dermatoscope), and additional types of optical viewing devices by presenting an optimal workflow on the imaging device 400 based on the type of optical viewing device associated with the instrument head 200.

As shown in FIG. 11, the method 1100 can include an operation 1102 of detecting the instrument head 200. In certain examples, the instrument head 200 is detected when attached to the imaging device 400. As described above, the imaging device 400 attaches to the eyepiece housing 218 of the instrument head 200 via the bracket 406. In some examples, the method 1100 does not include detecting the instrument head 200 such that operation 1102 is optional.

In some examples, the method 1100 can include preventing image capture by the imaging device 400 when attachment to the instrument head 200 is not detected in operation 1102. For example, the imaging device 400 is locked or blocked from capturing images unless the instrument head 200 is detected in operation 1102. When attachment to the instrument head 200 is detected, the imaging device 400 becomes unlocked or unblocked such that it is able to capture images. This feature can prevent use of the imaging device 400 for other purposes unrelated to capturing and displaying images from an optical viewing device 100. Additionally, this can prevent use of the imaging device 400 on unauthorized optical viewing devices. This feature can protect confidentiality of health information and provide theft deterrence.

Operation 1102 can include detecting the instrument head 200 based on the imaging performed by the camera 410 of the imaging device 400. As described above, the camera 410 is configured to capture images through the eyepiece 201 of the instrument head 200 when the housing 402 of the imaging device 400 is attached to the instrument head 200. The optics of the instrument head 200 differ based on whether the instrument head 200 belongs to the first type of optical viewing device 102, the second type of optical viewing device 104, or the third type of optical viewing device 106. As an illustrative example, the optics of the instrument head 200 can have a unique refractive index based on whether the instrument head 200 belongs to an otoscope, an ophthalmoscope, a dermatoscope, or other type of device. As another illustrative example, a lens component of the different types of optical viewing devices can include a notch, a label, a symbol, or other type of marking to facilitate detecting a type of the instrument head based on the images captured by the camera 410 of the imaging device 400 without affecting optical performance of the instrument head 200. In such examples, operation 1102 can include measuring or identifying one or more optical properties such as refractive index and/or markings to detect whether the imaging device 400 is attached to an instrument head 200 or not, and further to detect which type of optical viewing device 100 the instrument head 200 belongs to (i.e., an otoscope, an ophthalmoscope, a dermatoscope, or other type of optical viewing device).

In further examples, operation 1102 can include detecting the instrument head 200 based on physical contact with the instrument head 200. For example, the imaging device 400 can include a strain gauge or similar type of sensor inside the bracket 406 that can detect physical contact between the bracket 406 and the eyepiece housing 218.

In further examples, operation 1102 can include detecting the instrument head 200 based on a connection between the imaging device 400 and the instrument head 200. For example, the imaging device 400 can include one or more electrical contacts that complete a circuit when in contact with one or more electrical contacts on the instrument head 200. The instrument head 200 is detected when the one or more electrical contacts on the bracket 406 complete the circuit with the one or more electrical contacts on the instrument head 200. Operation 1102 can further include detecting which type of optical viewing device 100 the instrument head 200 belongs to (i.e., an otoscope, an ophthalmoscope, a dermatoscope, or other type of device) based on electrical signals received from the electrical circuit.

In further examples, the connection between the imaging device 400 and the instrument head 200 can be established via optical (e.g., infrared) or mechanical means, and detection of the instrument head 200 is based on detection of the optical or mechanical connection between the imaging device 400 and the instrument head 200.

In further examples, operation 1102 can include detecting the instrument head 200 based on machine-readable data provided by the instrument head 200. As described above, in some examples, the imaging device 400 can include a detector 408 that reads the machine-readable data from the identifier 220 on the instrument head 200.

In some examples, operation 1102 can include detecting the instrument head 200 based on a wireless signal received from the instrument head 200. For example, the identifier 220 of the instrument head 200 can include a wireless antenna that transmits a wireless signal that can be picked up by a wireless antenna (e.g., the detector 408) on the imaging device 400 when the imaging device 400 is attached to the instrument head 200. The wireless signal transmitted from the instrument head 200 to the imaging device 400 can include a radio frequency identification (RFID) signal, a near field communication (NFC) signal, a Bluetooth signal, a Wi-Fi signal, or type of wireless signal that can convey machine-readable data.

In some examples, the identifier 220 on the instrument head 200 is a passive wireless antenna and the detector 408 on the imaging device 400 is an active wireless antenna such that the identifier 220 on the instrument head 200 does not transmit the wireless signal unless activated by the detector 408 on the imaging device 400 such as when the imaging device 400 is attached to the instrument head 200. In some examples, the identifier 220 on the instrument head 200 is an RFID tag, an NFC tag, or similar type of wireless signal tag.

In further examples, operation 1102 can include detecting the instrument head 200 by reading a quick response (QR) code or other similar type of machine-readable label on the instrument head 200. For example, operation 1102 can include using the camera 410 of the imaging device 400 to read a machine-readable label (e.g., the identifier 220) on the instrument head 200 to detect the instrument head 200. In further examples, operation 1102 can include using a secondary camera (e.g., the detector 408) to read the machine-readable label (e.g., the identifier 220) on the instrument head 200. The machine-readable data (e.g., the identifier 220) when read by the imaging device 400 from the instrument head 200 can be further used to detect which type of optical viewing device 100 the instrument head 200 belongs to (i.e., an otoscope, an ophthalmoscope, a dermatoscope, or other type of device).

In further examples, operation 1102 can include detecting the instrument head 200 by using the camera 410 of the imaging device 400 to capture an image of the instrument head 200, and performing an analysis of the captured image to detect which type of optical viewing device 100 the instrument head 200 belongs to (i.e., an otoscope, an ophthalmoscope, a dermatoscope, or other type of device). In some examples, artificial intelligence or machine learning can be performed on the captured image to determine the type of the instrument head 200.

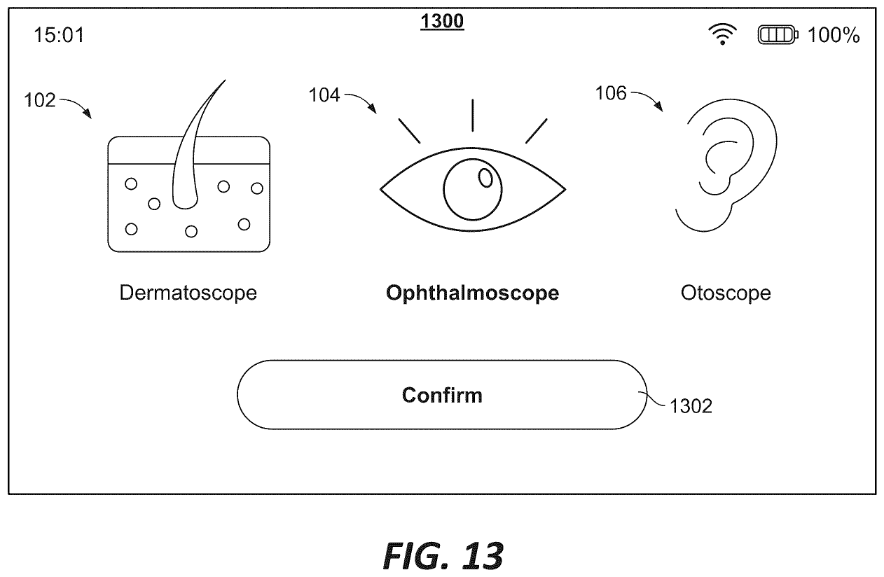

The method 1100 can include an operation 1104 of requesting confirmation of the type of optical viewing device 100 the instrument head 200 belongs to (i.e., an otoscope, an ophthalmoscope, a dermatoscope, or other type of device). For example, the imaging device 400 can display a graphical user interface on the display screen 404 that requests a user to confirm the type of optical viewing device 100. Examples of such a graphical user interface are shown in FIGS. 13, 27, and 36. In some examples, operation 1104 is optional.

As further shown in FIG. 11, the method 1100 includes an operation 1106 of determining a type of optical viewing device 100 the instrument head 200 belongs to based on the detection of the instrument head 200 in operation 1102 and/or a confirmation received from a user of the imaging device 400 based on the confirmation request in operation 1104. As an illustrative example, operation 1106 can include determining that the instrument head 200 belongs to the first type of optical viewing device 102 (e.g., an otoscope), or to the second type of optical viewing device 104 (e.g., an ophthalmoscope), or to the third type of optical viewing device 106 (e.g., a dermatoscope), or to another type of optical viewing device.

In some examples, the method 1100 can further include an operation 1108 of determining an environment of the imaging device 400. In some instances, particular regions, countries, or areas within a country may have unique regulations, naming conventions, languages, and customs related to the examinations performed by the type of optical viewing device determined in operation 1106. In some further instances, a particular type of medical facility, or a department or unit within a medical facility, may influence the examinations performed by the type of optical viewing device determined in operation 1106. For example, the optical viewing device may be used differently when located in an emergency department where a broad range of patients are examined than when being used in a pediatric ward of a hospital where a specific patient population is examined (i.e., children) or a particular type of examination is performed (i.e., checking for acute otitis media in the ear). Also, the type of optical viewing device determined in operation 1106 may be used differently in a teaching or learning environment than when being used to examine patients in a clinical environment.

Accordingly, in some examples, operation 1108 can include determining a geographic location of the imaging device 400 such as a region, a country, or an area within a country. Additionally, or alternatively, operation 1108 can include determining whether the imaging device 400 is located in a particular type of medical facility such as a hospital, a nursing home, or other type of facility, or is located in a particular unit or department within a medical facility such as an emergency department, a pediatric ward, or other type of department or unit within a hospital. Additionally, or alternatively, operation 1108 can include determining whether the imaging device 400 is located in a training facility, or is being used in a clinical environment.

Operation 1108 can include determining the environment of the imaging device 400 based on an Internet Protocol (IP) address of the network 5052. Additionally, or alternatively, operation 1108 can include determining the environment of the imaging device 400 using one or more types of geolocation or geopositioning techniques such as cell tower triangulation, satellite-based radio navigation such as by using the Global Positioning System (GPS), Wi-Fi positioning, and hybrid geopositioning techniques.

In some examples, the method 1100 can include an operation 1110 of determining a user of the type of optical viewing device 100 determined in operation 1106. In some instances, the user may influence the examinations performed by the type of optical viewing device determined in operation 1106. For example, the type of optical viewing device 100 determined in operation 1106 may be used differently when being used by a trainee during a training mode than when being used by a medical professional in a clinical mode. As another example, the type of optical viewing device 100 determined in operation 1106 may be used differently when being used by a specialist (e.g., ophthalmologist) than when being used by a general practitioner or by a medical professional having lower credentials (e.g., a nurse practitioner or physician assistant).

Operation 1110 can include determining the user by using the camera 410 to scan a machine-readable code associated with the user such as an employee ID barcode attached to an item worn by the user such as a lanyard, bracelet, and the like. Alternatively, or additionally, operation 1112 can include determining the user based on one or more credentials entered by the user on the imaging device 400 such as username and password.

In some examples, the method 1100 can further include an operation 1112 of determining a patient who is undergoing examination by the type of optical viewing device 100 determined in operation 1106. In some instances, a patient may influence the examinations performed by the type of optical viewing device determined in operation 1106. For example, the type of optical viewing device 100 determined in operation 1106 may be used differently when being used to examine a geriatric patient than when being used to examine a pediatric patient because the optical viewing device 100 would be used to detect conditions prevalent in geriatric patient populations which may differ from conditions prevalent in pediatric patient populations.

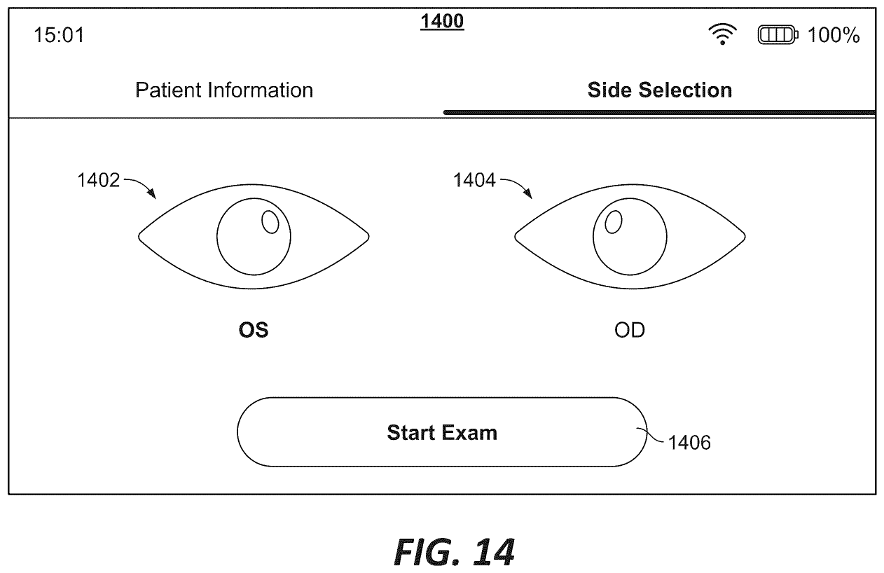

Operation 1112 can include determining the patient such as by using the camera 410 to scan a barcode associated with the patient such as a barcode attached to a wristband worn by the patient, or a barcode attached to a file of the patient. Alternatively, or additionally, operation 1112 can include determining the patient by presenting a graphical user interface that allows a user of the imaging to search for the patient (e.g., see FIG. 14), and thereafter receiving a selection of the patient on the graphical user interface displayed on the imaging device 400.

The method 1100 further includes an operation 1114 of presenting a workflow on the imaging device 400 based on the type of optical viewing device 100 determined in operation 1106. The workflow presented in operation 1114 can include one or more tools for annotating one or more frames selected from a video of an anatomy captured by the imaging device 400. For example, the workflow can include tools such as one or more predefined annotations associated with a type of anatomy typically examined by the optical viewing device 100 determined in operation 1106. The one or more predefined annotations are selectable on the display screen 404 to indicate presence of a condition or disease state associated with the type of anatomy. The workflow can also include tools such as one or more filters based on the type of anatomy typically examined by the optical viewing device 100 determined in operation 1106. For example, filters such as a grayscale filter, a high-contrast filter, and a red-free filter can be pre-selected for use on the imaging device based on the optical viewing device 100 determined in operation 1106. The workflow can also include tools such as a ruler superimposed on a selected frame of an anatomy, and the ruler is scaled based on the type of anatomy typically examined by the optical viewing device 100 determined in operation 1106. The ruler can provide a reference for one or more anatomical features included in the selected frame.

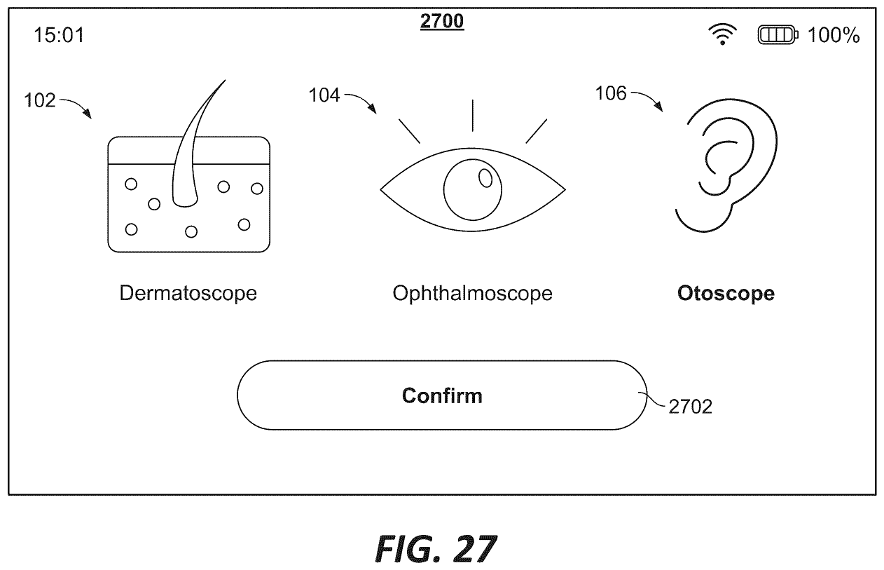

Operation 1114 can include presenting a first type of workflow that is optimal for the first type of optical viewing device 102 (e.g., an otoscope) when operation 1106 determines the instrument head 200 belongs to the first type of optical viewing device 102. Illustrative examples of the first type of workflow are shown in FIGS. 27-35.

Operation 1114 can include presenting a second type of workflow that is optimal for the second type of optical viewing device 104 (e.g., an ophthalmoscope) when operation 1106 determines the instrument head 200 belongs to the second type of optical viewing device 104. Illustrative examples of the second type of workflow are shown in FIGS. 13-26.

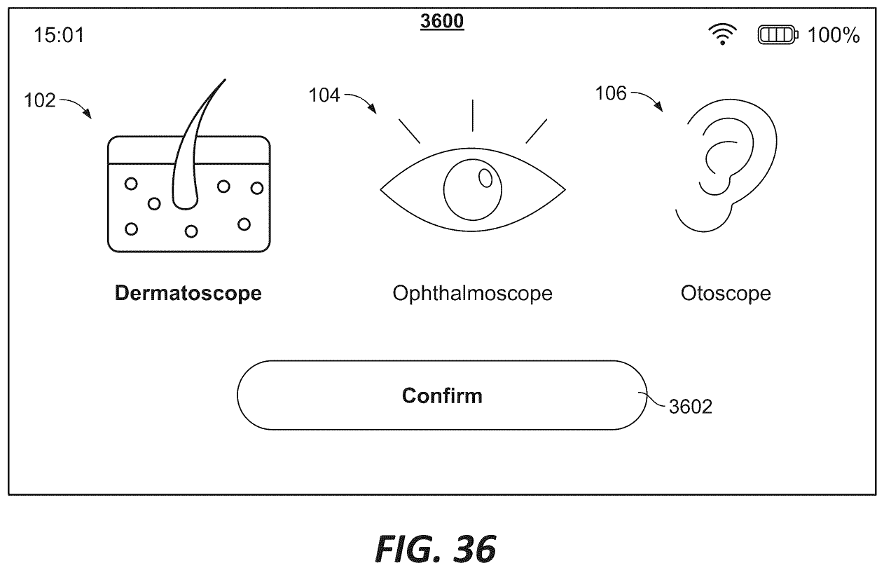

Operation 1114 can include presenting a third type of workflow that is optimal for the third type of optical viewing device 106 (e.g., a dermatoscope) when operation 1106 determines that the instrument head 200 belongs to the third type of optical viewing device 106 (e.g., a dermatoscope). Illustrative examples of the third type of workflow are shown in FIGS. 36-49. While three different types of workflows are discussed herein, the method 1100 can include presenting additional types of workflows for additional types of optical viewing devices.

In addition to presenting customized workflows on the imaging device 400, operation 1114 can further include presenting a preset zoom on the imaging device 400 based on an anatomy typically examined by the optical viewing device 100 determined in operation 1106. For example, different preset zooms can be presented on the imaging device 400 based on whether the imaging device 400 is attached to an ophthalmoscope, an otoscope, a dermatoscope, or other type of optical viewing device. Each preset zoom is optimal for imaging a particular type of anatomy such as an eye, an ear, or a skin surface using a particular scope. In further examples, different image properties like brightness and focus can be presented on the imaging device 400 based on whether the imaging device 400 is attached to an ophthalmoscope, an otoscope, a dermatoscope, or other type of optical viewing device.

In some examples, operation 1114 includes modifying the workflow based on one or more of the environment of the imaging device 400 detected in operation 1108, the user detected in operation 1110, and the patient detected in operation 1112.

As an illustrative example, operation 1114 can include modifying the first, second, or third workflows based on the environment detected in operation 1108. For example, operation 1114 can include genericizing the workflows such as by disabling certain features displayed on the imaging device 400 when the type of optical viewing device 100 determined in operation 1106 is detected as being used in the emergency department of a hospital where a broad range of patients are examined. Alternatively, operation 1114 can include making the workflows more specialized by enabling certain features on the imaging device 400 when the type of optical viewing device 100 determined in operation 1106 is detected as being used in a unit having a targeted patient population (e.g., pediatrics). Additional examples are contemplated.

As another example, operation 1114 can include modifying the first, second, or third workflows based on the user detected in operation 1108. For example, operation 1114 can include genericizing the workflows such as by disabling certain features displayed on the imaging device 400 when the user is detected as being a general practitioner or as having a lower level of experience or expertise. Alternatively, operation 1114 can include making the workflows more specialized by enabling certain features on the imaging device 400 when the user is detected as being a specialist such as an ophthalmologist. Additional examples are contemplated.

As another example, operation 1114 can include modifying the first, second, or third workflows based on the patient detected in operation 1112. For example, operation 1114 can include modifying the workflows by enabling and/or disabling certain features displayed on the imaging device 400 when the patient belongs to one type of patient population (e.g., geriatric patients), and can include modifying the workflows by enabling and/or disabling certain features displayed on the imaging device 400 when the patient belongs to another type of patient population (e.g., pediatric patients). Additional examples are contemplated.

In such examples where operation 1114 includes modifying the first, second, or third workflows based on the environment, the user, or the patient, the imaging device 400 can operate under an advanced mode where advanced features are enabled, or can alternatively operate under a basic mode where advanced features are disabled. This can provide more efficient workflows on the imaging device 400 for faster learning by new or inexperienced users.

Further, as described above, a training mode can be enabled on the imaging device 400 such as when being used by a trainee, and a clinical mode can be enabled on the imaging device when being used to clinically assess a patient. When in the clinical mode, the workflows prevent the user from storing images on the imaging device 400 to limit access to protected health information (PHI), whereas when in the training mode, the workflows allow temporary storage of images on the imaging device 400 for learning and training purposes.

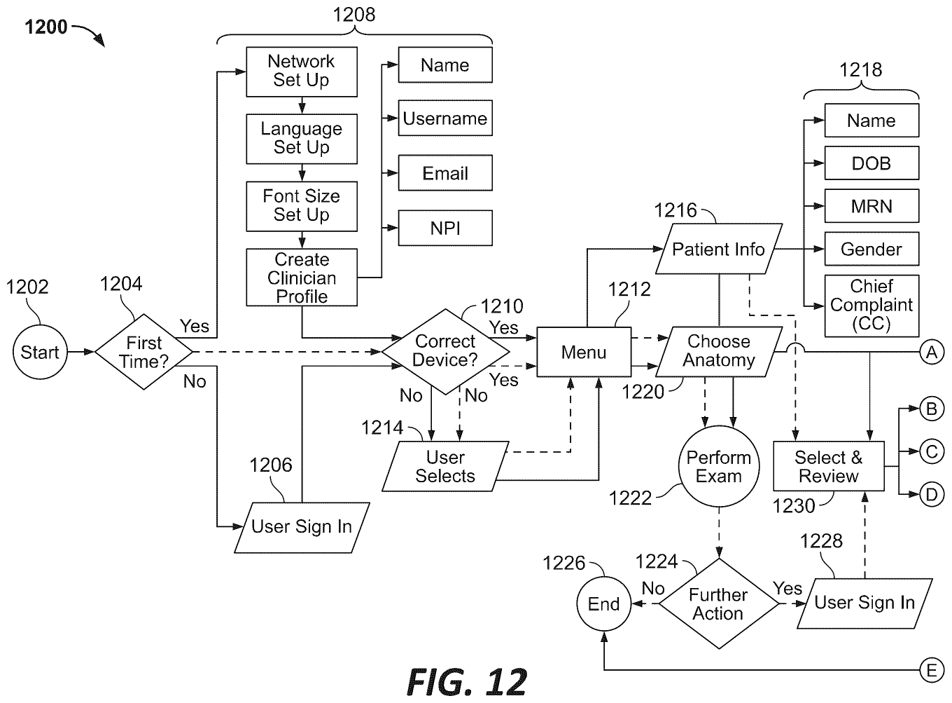

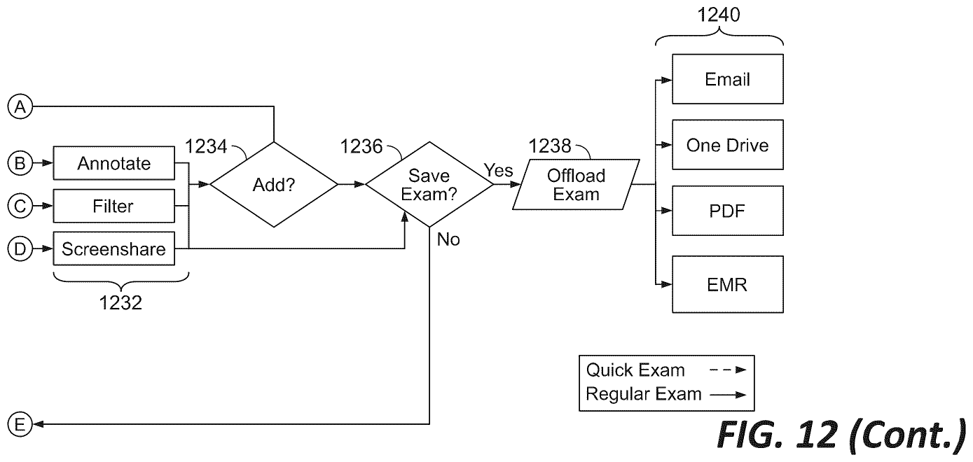

FIG. 12 illustrates an example of a workflow 1200 that can be presented on the imaging device 400. In certain examples, the workflow 1200 is presented in accordance with the operations of the method 1100 described above. The workflow 1200 starts at an operation 1202 such as when the imaging device 400 is turned on by a user.

The workflow includes an operation 1204 of determining whether the imaging device 400 is being used for a first time by the user. When the imaging device 400 is not being used for the first time by the user (i.e., “No” in operation 1204), the workflow 1200 can proceed to an operation 1206 of requesting the user to enter their sign-in credentials such as a username and a password. When the imaging device 400 is being used for the first time by the user (i.e., “Yes” in operation 1204), the workflow 1200 can proceed to an operation 1208 of requesting the user set up a profile on the imaging device 400. Operation 1208 can include requesting the user to set up the network 5052 for connecting the imaging device 400 to the external system 600, requesting the user to set their language preferences, requesting the user to set their font size preferences, and requesting the user to create a user profile that can include user information such as the user's name, username, email, national provider identifier (NPI), and other information.

After completion of operation 1206 or operation 1208, the workflow 1200 proceeds to an operation 1210 of requesting confirmation that the determination of the type of optical viewing device 100 (see operation 1106 of the method 1100) is correct. Alternatively, the workflow 1200 can include a quick exam protocol in which operations 1206 and 1208 are skipped, and the workflow 1200 proceeds directly to operation 1210.

Operation 1210 can include presenting a graphical user interface, such as the graphical user interfaces 1300, 2700, and 3600 respectively shown in FIGS. 13, 27, and 36, that requests the user to confirm the type of optical viewing device 100 identified by the imaging device 400. As shown in FIGS. 13, 27, and 36, the type of optical imaging device identified by the imaging device 400 is highlighted as a dermatoscope, an ophthalmoscope, or an otoscope, and is confirmed by receiving a selection of a confirm icon 1302, 2702, 3602.

When the type of optical viewing device 100 is confirmed as correct (i.e., “Yes” in operation 1210), the workflow 1200 proceeds to an operation 1212 of displaying a graphical user interface that allows the user to select a patient and an anatomy for examination. When the type of optical viewing device 100 is not confirmed as correct (i.e., “No” in operation 1210), the workflow 1200 proceeds to an operation 1214 of receiving a selection of the correct type of optical viewing device 100. Thereafter, the workflow proceeds to operation 1212.

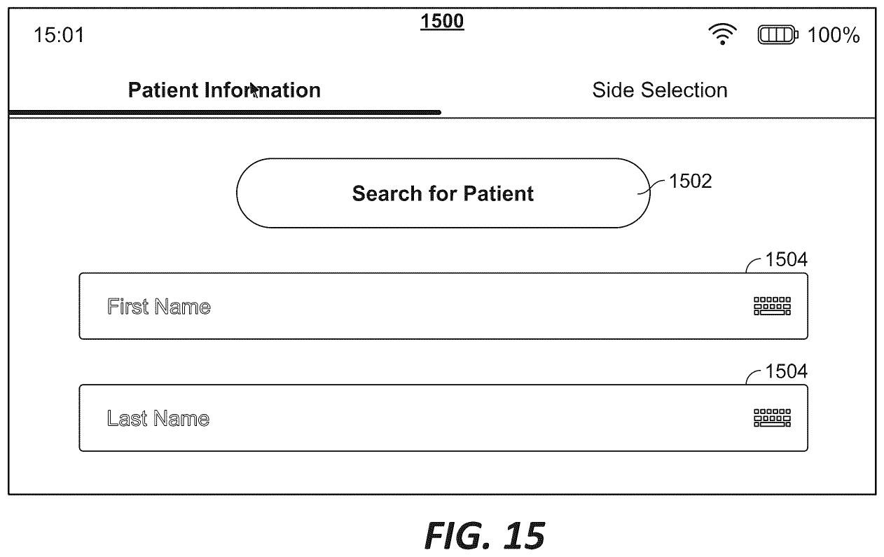

Operation 1212 can include displaying a graphical user interface 1500 (see FIG. 15) allowing the user to search for an existing patient such as by entering patient information 1218 into one or more text fields 1504 and selecting a search for patient icon 1502. When the user selects a text field 1504, a keyboard is displayed on the display screen 404 of the imaging device 400 allowing the user to type the relevant patient information in the text field. Examples of the patient information 1218 include patient name, date of birth, medical record number (MRN), gender, and complaint. In examples where the patient is not an existing patient, the graphical user interface 1500 allows the user to enter the patient information 1218 for a new patient.

As further shown in FIG. 12, the workflow 1200 includes an operation 1220 of receiving a selection of an anatomy for examination. As an illustrative example, when the type of optical viewing device is determined to be an ophthalmoscope, operation 1220 includes displaying a graphical user interface 1400 (see FIG. 14) that includes first and second options 1402, 1404 allowing the user to select the left eye (OS) or the right eye (OD), and thereafter select a start exam icon 1406 to start an examination of the left eye (OS) or the right eye (OD).

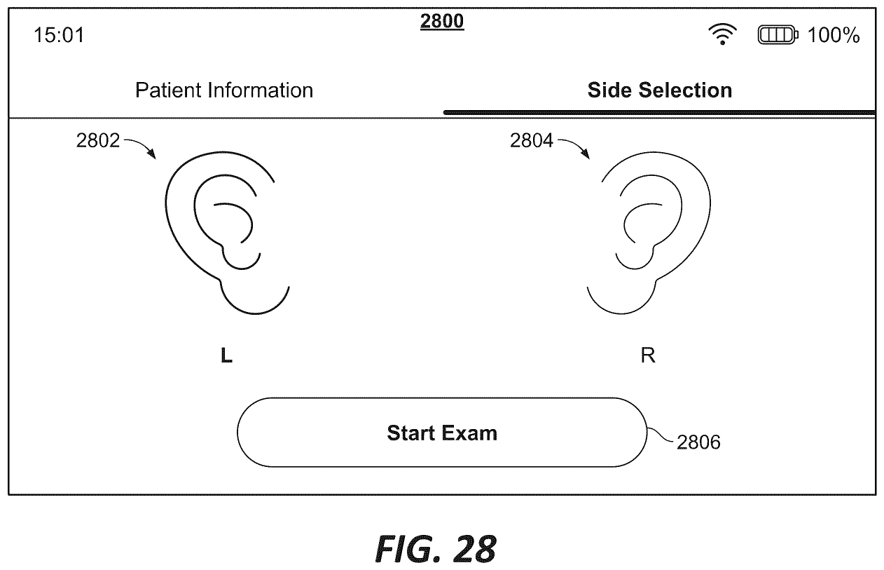

As another example, when the type of optical viewing device is determined to be an otoscope, operation 1220 includes displaying a graphical user interface 2800 (see FIG. 28) that has first and second options 2802, 2804 allowing the user to select the left ear or the right ear, and thereafter select a start exam icon 2806 to start an examination of the left ear or right ear.

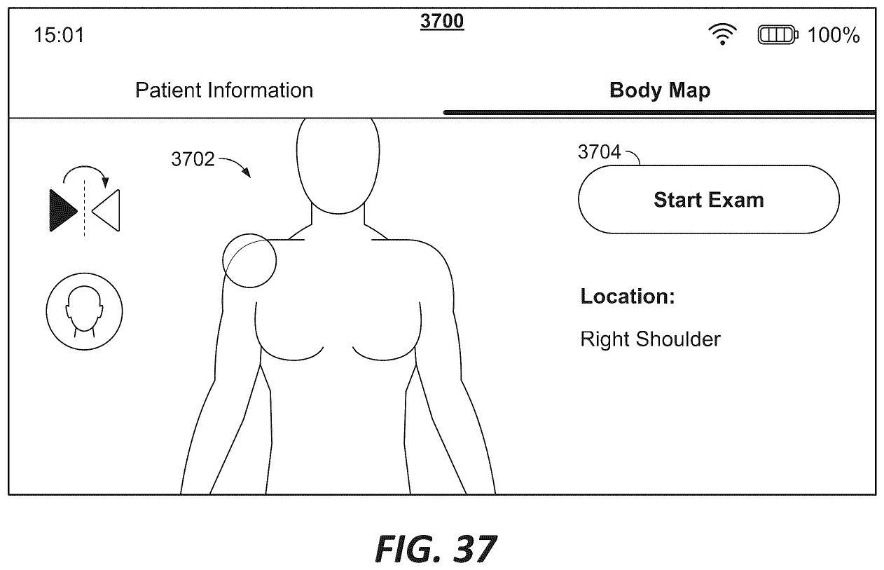

As another example, when the type of optical viewing device is determined to be a dermatoscope, operation 1220 includes displaying a graphical user interface 3700 (see FIG. 37) that has a body profile 3702 for the user to select an area (e.g., right shoulder), and to select a start exam icon 3704 to start an examination of the selected area on the body profile 3702.

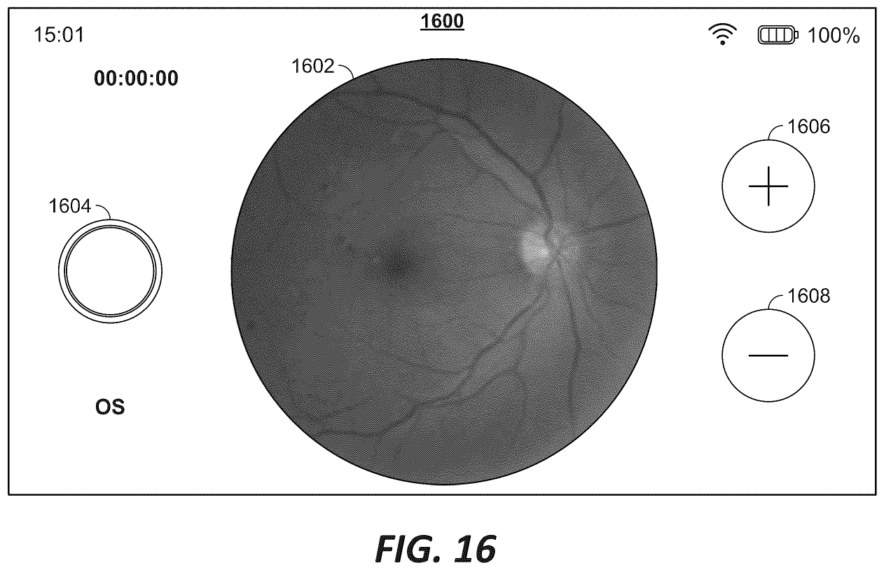

The workflow 1200 includes to an operation 1222 of displaying a graphical user interface that allows the user to capture a video of the anatomy selection received in operation 1220. As an example, operation 1222 can include displaying a graphical user interface 1600 (see FIG. 16) when the type of optical viewing device is an ophthalmoscope. The graphical user interface 1600 allows the user to record a video of the left eye (OS) or the right eye (OD) shown inside a display area 1602 by selecting a capture icon 1604. The graphical user interface 1600 includes a zoom-in icon 1606 and a zoom-out icon 1608 that allow the user to zoom in and out the left or right eye displayed in the display area 1602, which is captured by the camera 410 of the imaging device 400. As described above, the camera 410 captures the video through the eyepiece 201 of the second type of optical viewing device 104 (e.g., ophthalmoscope).

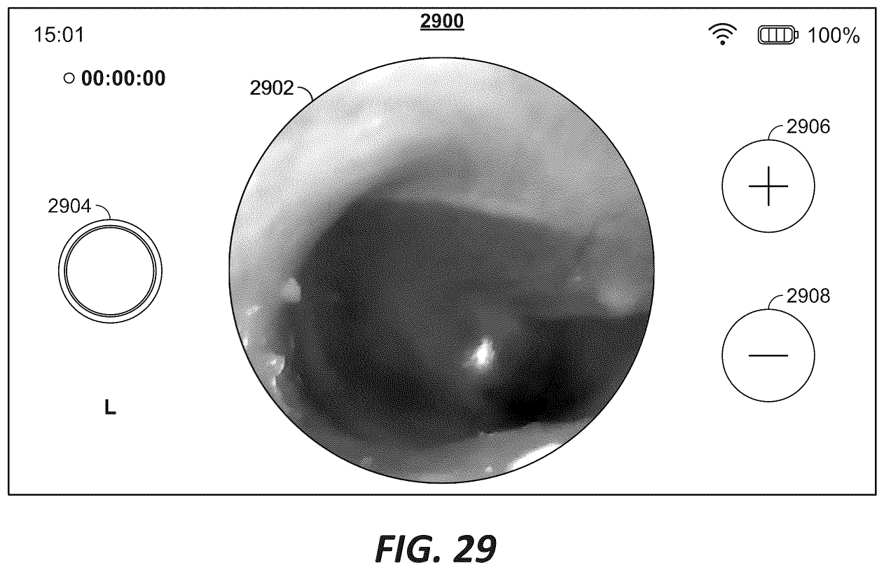

As another example, operation 1222 can include displaying a graphical user interface 2900 (see FIG. 29) when the type of optical viewing device is an otoscope. The graphical user interface 1600 allows the user to record a video of the left ear drum or the right ear drum shown inside a display area 2902 by selecting a capture icon 2904. The graphical user interface 2900 includes a zoom-in icon 2906 and a zoom-out icon 2908 that allow the user to zoom in and out the left or right ear drum displayed in the display area 2902, which is captured by the camera 410 of the imaging device 400. As described above, the camera 410 captures the video through the eyepiece 201 of the first type of optical viewing device 102 (e.g., otoscope).

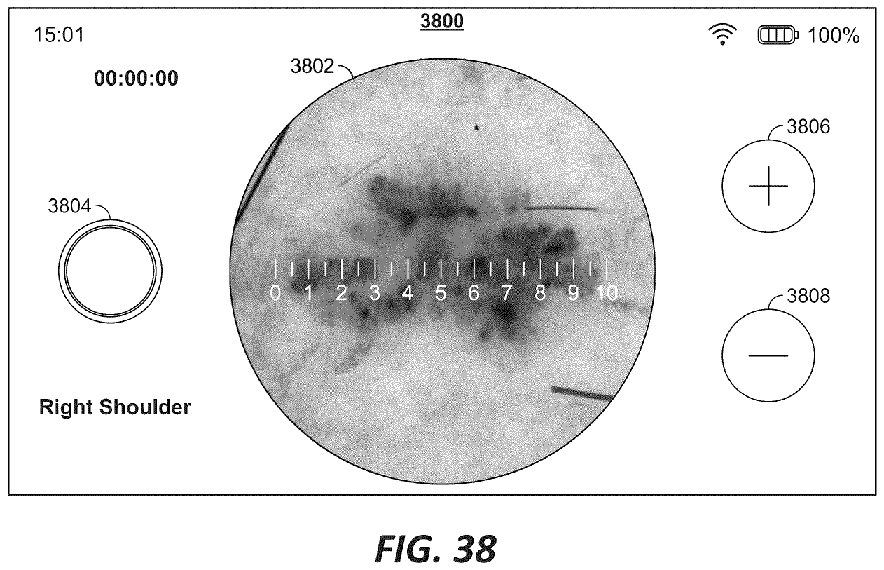

As another illustrative example, operation 1222 can include displaying a graphical user interface 3800 (see FIG. 38) when the type of optical viewing device is a dermatoscope. The graphical user interface 3800 allows the user to record a video of a skin area shown inside a display area 3802 by selecting a capture icon 3804. The graphical user interface 3800 includes a zoom-in icon 3806 and a zoom-out icon 3808 that allow the user to zoom in and out the skin area displayed in the display area 3802, which is captured by the camera 410 of the imaging device 400. As described above, the camera 410 captures the video through the eyepiece 201 of the third type of optical viewing device 106 (e.g., dermatoscope).

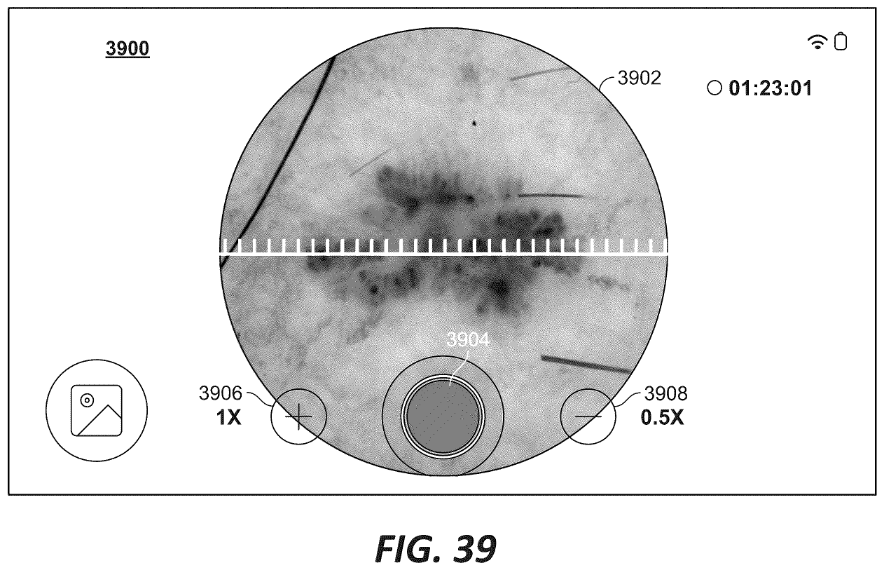

FIG. 39 provides another example of a graphical user interface 3900 that can be displayed on the imaging device 400 when attached to a dermatoscope. The graphical user interface 3900 allows the user to record a video of a skin area shown inside a display area 3902 by selecting a capture icon 3904. The graphical user interface 3900 includes a zoom-in icon 3906 and a zoom-out icon 3908 that allow the user to zoom in and out the video of the skin area captured by the camera 410 of the imaging device 400 and displayed in the display area 3902.

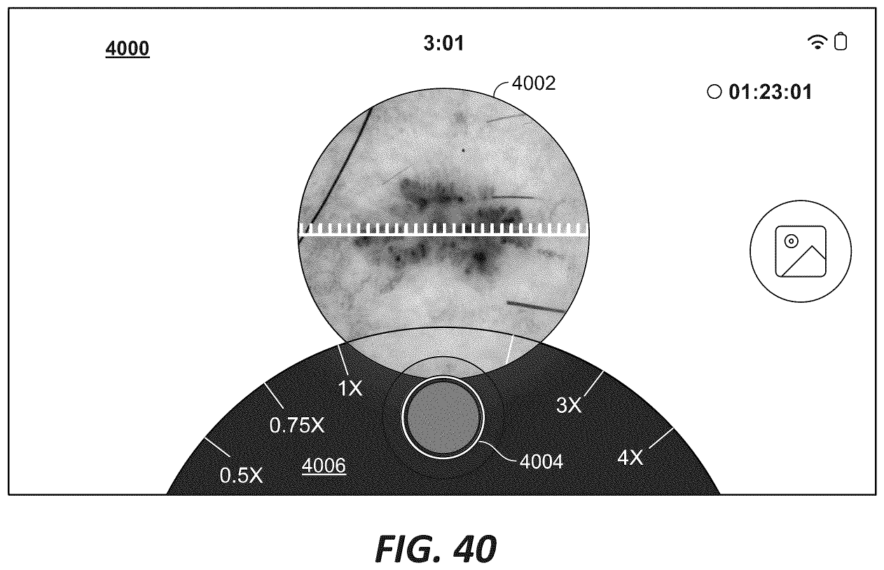

FIG. 40 provides another example of a graphical user interface 4000 that can be displayed on the imaging device 400 when attached to a dermatoscope. The graphical user interface 4000 similarly allows the user to record a video of a skin area shown inside a display area 4002 by selecting a capture icon 4004. The graphical user interface 4000 includes a zoom wheel 4006 that can be rotated in a clockwise direction to zoom out, and that can be rotated in a counterclockwise direction to zoom in the video of the skin area captured by the camera 410 of the imaging device 400 and displayed in the display area 4002.

Once the video of the anatomy is captured in operation 1222, the workflow 1200 includes an operation 1230 of displaying a graphical user interface that allows the user to review the video and to select one or more frames from the video for further analysis and storage in an electronic medical record of the patient, as will be described in more detail further below.

In examples where the quick exam protocol is implemented on the imaging device 400, the workflow 1200 can proceed to an operation 1224 of determining whether there is a further action. When there is no further action (i.e., “No” in operation 1224), the workflow 1200 ends at operation 1226. In such examples, the video captured in operation 1222 can be discarded or deleted. In such examples, the workflow 1200 is performed as part of a training session.

In the quick exam protocol, when there is a further action (i.e., “Yes” in operation 1224), the workflow 1200 can proceed to an operation 1228 of requesting the user to enter their credentials such as their username and password. Once the user's credentials are entered such that the user is authenticated, the workflow 1200 can proceed to the operation 1230.

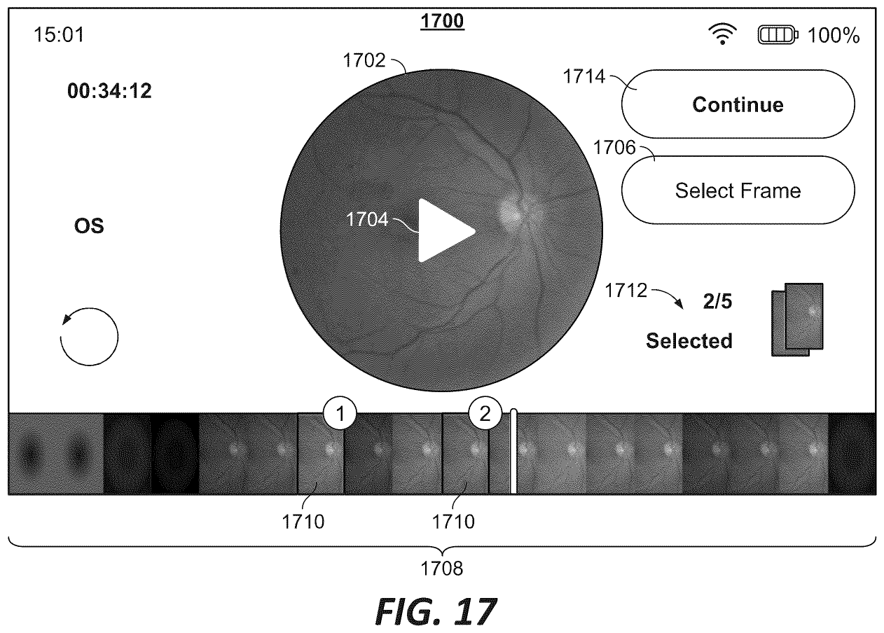

Operation 1230 can include displaying a graphical user interface 1700 (see FIG. 17) when the type of optical viewing device is an ophthalmoscope. The graphical user interface 1700 includes a display area 1702 that allows the user to playback the video of the left eye (OS) or the right eye (OD) by selecting a playback icon 1704. The graphical user interface 1700 includes a select frame icon 1706 that allows the user to select one or more frames 1710 from a plurality of frames 1708 displayed at the bottom of the graphical user interface 1700. The graphical user interface 1700 can display in an area 1712 the number of frames selected by the user (e.g., 2frames) out of a maximum number of frames allowed (e.g., 5 frames).

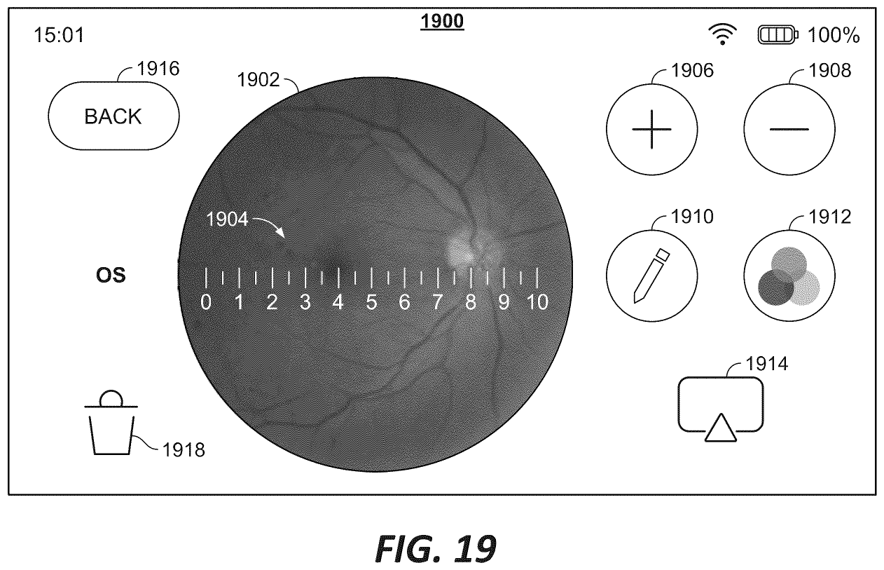

Once the user is satisfied with the selected frames from the video of the left eye (OS) or the right eye (OD), the user can select a continue icon 1714. Thereafter, the workflow 1200 can proceed to an operation 1232 allowing the user to customize the one or more frames selected in operation 1230. For example, operation 1232 can include displaying a graphical user interface 1800 that displays one or more frames 1802 selected from the videos of the left eye (OS) and/or the right eye (OD). The user can select an edit icon 1804 which causes a display of a graphical user interface 1900 (see FIG. 19) that allows the user to edit a selected frame.

The graphical user interface 1900 includes a display area 1902 of the selected frame of the left eye (OS) or the right eye (OD). The display area 1902 includes a ruler 1904 superimposed on the selected frame of the left eye (OS) and/or the right eye (OD). The ruler 1904 is scaled based on the anatomy displayed in the display area 1902, and provides a reference for one or more anatomical features included in the selected frame in the display area 1902. The graphical user interface 1900 includes a return icon 1916 that when selected causes the workflow 1200 to return back to the graphical user interface 1800 that displays one or more frames 1802. Also, the graphical user interface 1900 includes a delete icon 1918 that when selected causes the workflow 1200 to delete the selected frame of the left eye (OS) or the right eye (OD).

The graphical user interface 1900 has a zoom-in icon 1906 and a zoom-out icon 1908 that can be selected to zoom in or out the selected frame of the left eye (OS) or the right eye (OD). The graphical user interface 1900 includes an annotations icon 1910 that when selected allows the user to add one or more annotations to the frame displayed in the display area 1902.

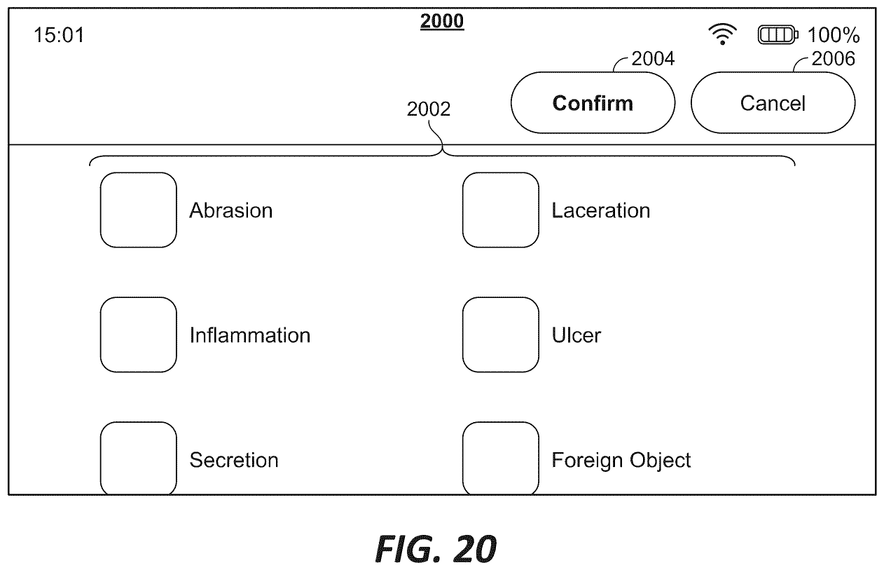

As an illustrative example, the annotations icon 1910 when selected by the user causes the workflow 1200 to display a graphical user interface 2000 (see FIG. 20) that can include one or more predefined annotations 2002 based on the anatomy displayed by the frame. In examples when the imaging device 400 is attached to an ophthalmoscope such that the anatomy is that of the left or right eyes, the one or more predefined annotations 2002 can include selectable options such as abrasion, laceration, inflammation, ulcer, secretion, and foreign object which may be relevant to a physical assessment of the left and right eyes. The graphical user interface 2000 may also allow the user to type one or more customized annotations. Once the user is satisfied with the annotations, the user can select a confirm icon 2004 to return to the graphical user interface 1900. Also, the user may select a cancel icon 2006 to cancel selections of the one or more annotations and return to the graphical user interface 1900.

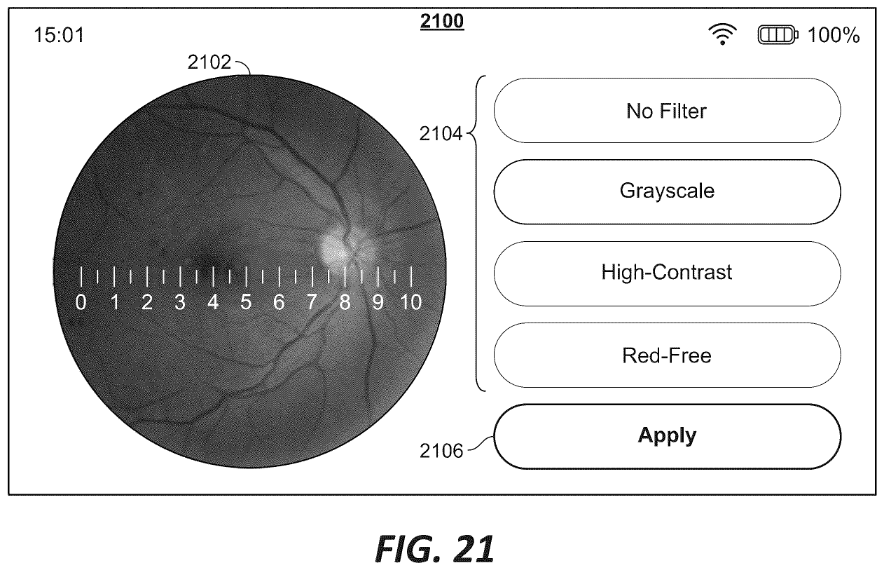

Referring back to FIG. 19, the graphical user interface 1900 can include a filter icon 1912 that when selected allows the user to add one or more filters to the frame displayed in the display area 1902. The filter icon 1912 when selected by the user causes the workflow 1200 to display a graphical user interface 2100 (see FIG. 21) that can include one or more filters 2104 that are displayed based on the anatomy displayed inside a display area 2102. In examples when the imaging device 400 is attached to an ophthalmoscope such that the anatomy is that of the left or right eyes, the one or more filters 2104 can include no filter, grayscale filter, high-contrast filter, and red-free filter. Once the user is satisfied with a selected filter or no filter, the user can select an apply icon 2106 to return to the graphical user interface 1900.

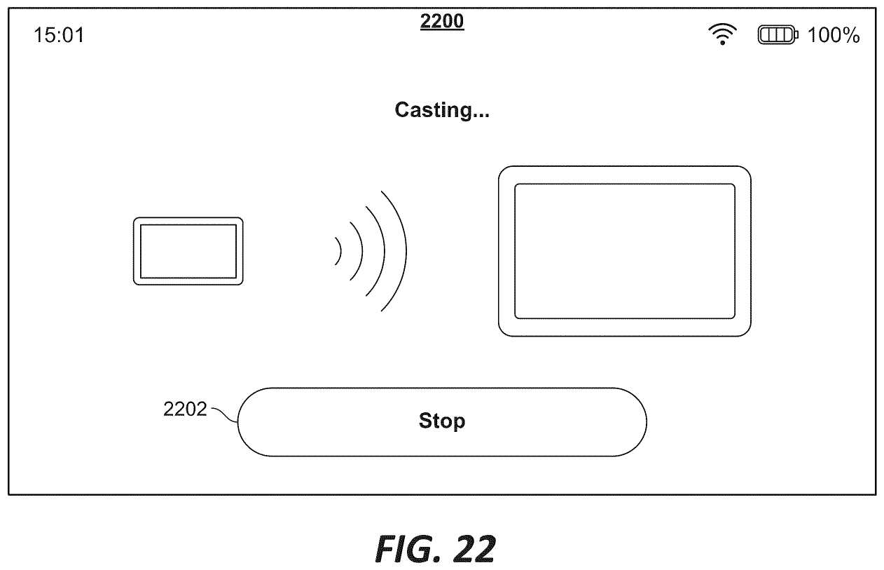

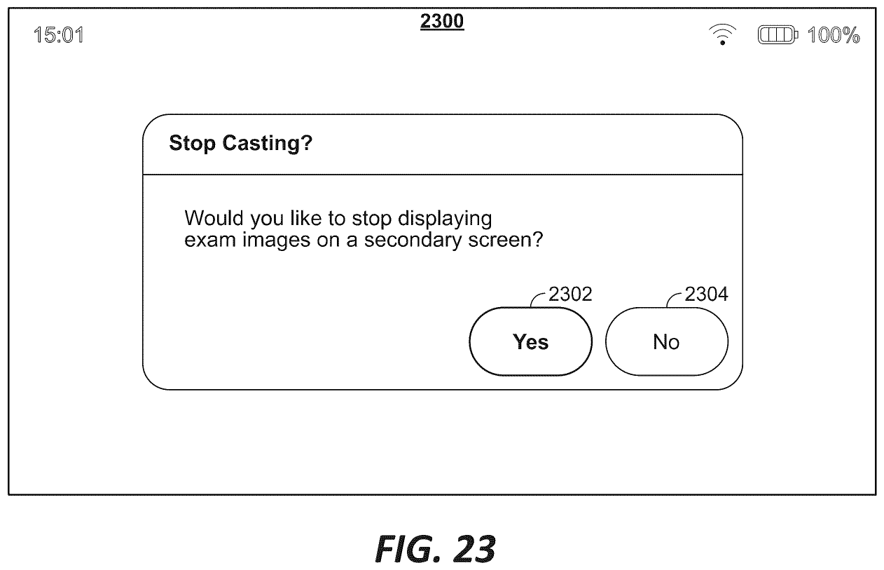

As further shown in FIG. 19, the graphical user interface 1900 can include a casting icon 1914 that causes the frame displayed in the display area 1902 to be streamed or casted to another display device or monitor. When the frame is being streamed or casted to another display device or monitor, a graphical user interface 2200 (see FIG. 22) can be displayed on the display screen 404 of the imaging device 400. The graphical user interface 2200 includes a stop icon 2202 that when selected can cause the workflow 1200 to display a graphical user interface 2300 (see FIG. 23) that requests confirmation to stop streaming or casting the frame on the other display device or monitor such as by selecting a yes icon 2302 or selecting a no icon 2304.

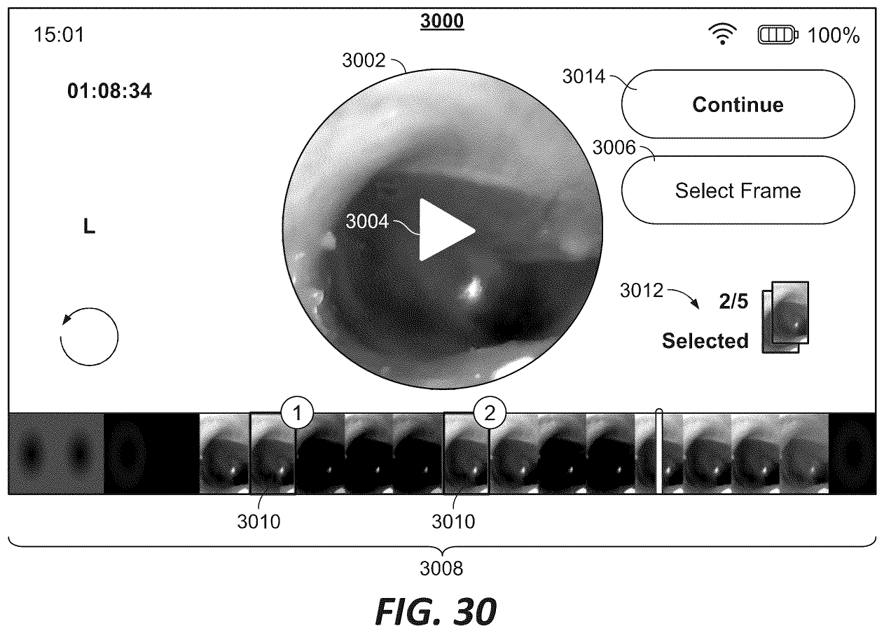

Referring back to FIG. 12, operation 1230 can include displaying a graphical user interface 3000 (FIG. 30) when the type of optical viewing device is an otoscope. The graphical user interface 3000 includes a display area 3002 that allows the user to playback the video of the left ear or the right ear by selecting a playback icon 3004. The graphical user interface 3000 includes a select frame icon 3006 that allows the user to select one or more frames 3010 from a plurality of frames 3008 displayed at the bottom of the graphical user interface 3000. The graphical user interface 3000 can display in an area 3012 the number of frames selected by the user (e.g., 2 frames) out of a maximum number of frames allowed (e.g., 5 frames).

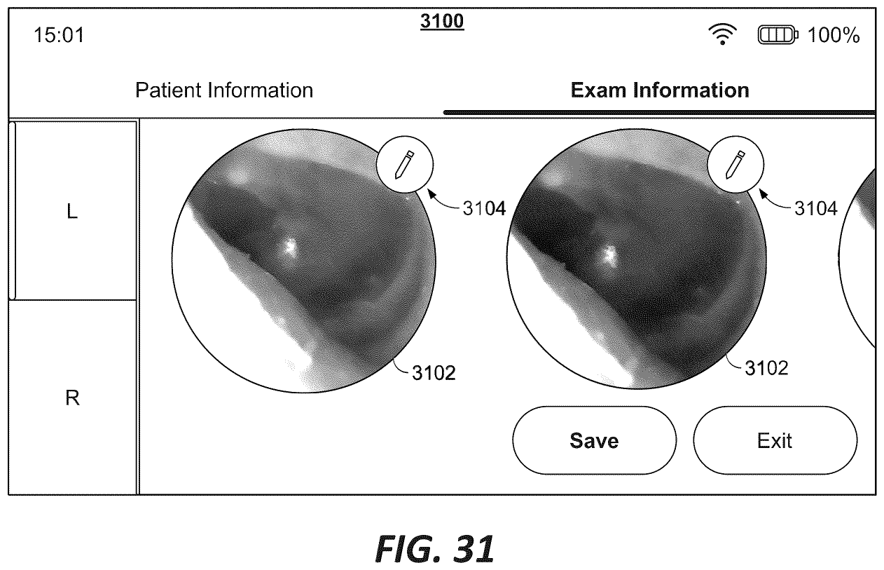

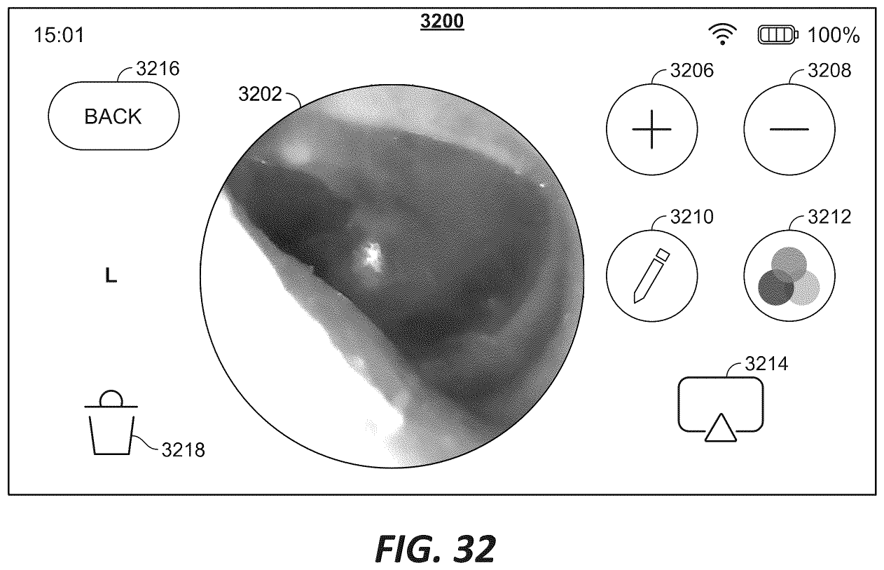

Once the user is satisfied with the selected frames from the video of the left ear or the right ear, the user can select a continue icon 3014. Thereafter, the workflow 1200 can proceed to the operation 1232 allowing the user to customize the one or more frames selected in operation 1230. For example, operation 1232 can include displaying a graphical user interface 3100 (see FIG. 31) that displays one or more frames 3102 selected from the videos of the left ear and/or the right ear. The user can select an edit icon 3104 which causes a display of a graphical user interface 3200 (see FIG. 32) that allows the user to edit a selected frame of the left or right ear.

The graphical user interface 3200 includes a display area 3202 of the selected frame of the left ear or the right ear. The graphical user interface 3200 includes a return icon 3216 that when selected causes the workflow 1200 to return back to the graphical user interface 3100 that displays one or more frames 3102. The graphical user interface 3200 includes a delete icon 3218 that when selected causes the workflow 1200 to delete the selected frame of the left or right ear.

The graphical user interface 3200 has a zoom-in icon 3206 and a zoom-out icon 3208 that can be selected to zoom in or out the selected frame of the left ear or the right ear. The graphical user interface 3200 includes an annotations icon 3210 that when selected allows the user to add one or more annotations to the frame displayed in the display area 3202.

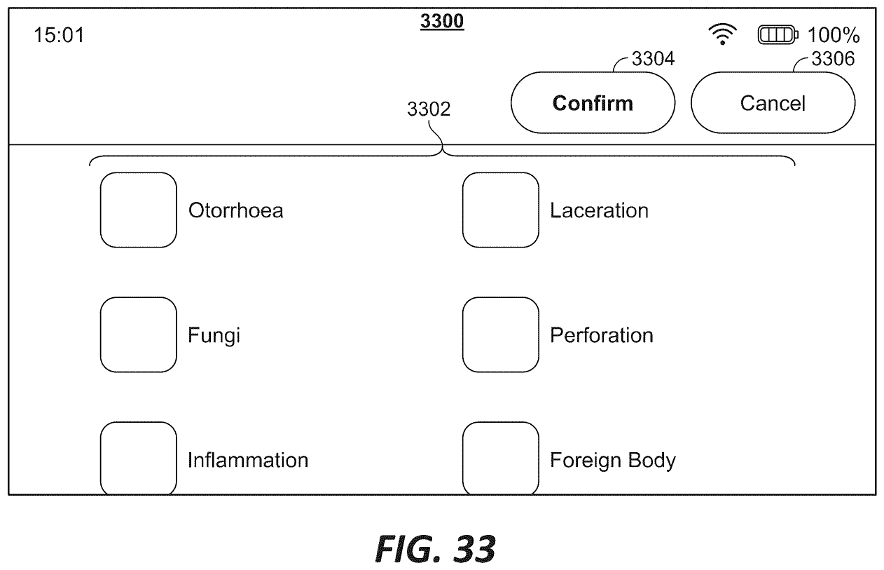

As an illustrative example, the annotations icon 3210 when selected by the user causes the workflow 1200 to display a graphical user interface 3300 (see FIG. 33) that can include one or more predefined annotations 3302 based on the anatomy displayed by the frame. In examples when the imaging device 400 is attached to an otoscope such that the anatomy is that of the left or right ear, the one or more predefined annotations 3302 can include selectable options such as otorrhea, laceration, fungi, perforation, inflammation, and foreign body which may be relevant to a physical assessment of the left and right ears. The graphical user interface 3300 may also allow the user to type one or more customized annotations. Once the user is satisfied with the annotations, the user can select a confirm icon 3304 to return to the graphical user interface 3200. Also, the user may select a cancel icon 3306 to cancel selections of the one or more annotations and return to the graphical user interface 3200.

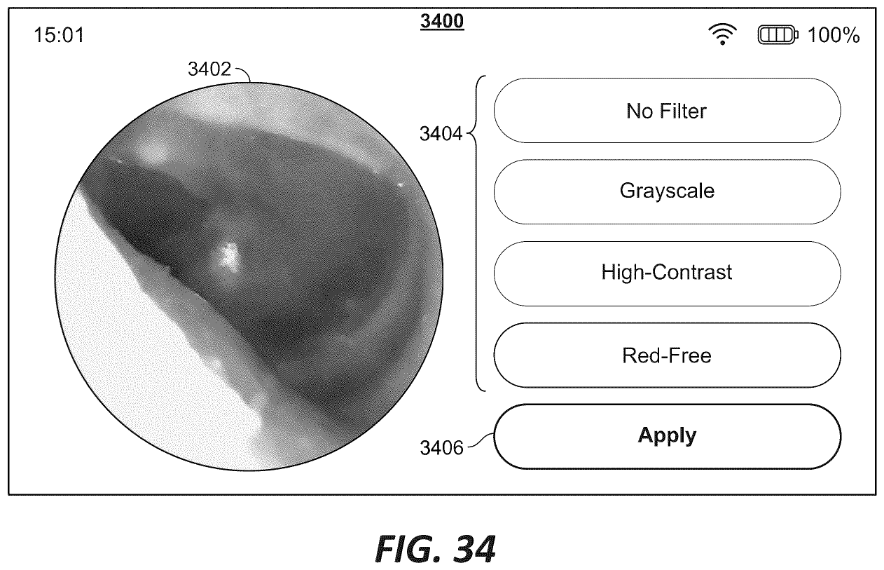

Referring back to FIG. 32, the graphical user interface 3200 can include a filter icon 3212 that when selected allows the user to add one or more filters to the frame displayed in the display area 3202. The filter icon 3212 when selected by the user causes the workflow 1200 to display a graphical user interface 3400 (see FIG. 34) that can include one or more filters 3404 that are displayed based on the anatomy displayed inside a display area 3402. In examples when the imaging device 400 is attached to an otoscope such that the anatomy is that of the left or right ear, the one or more filters 3404 can include no filter, grayscale filter, high-contrast filter, and red-free filter. Once the user is satisfied with a selected filter or no filter, the user can select an apply icon 3406 to return to the graphical user interface 3200.

As further shown in FIG. 32, the graphical user interface 3200 can include a casting icon 3214 that causes the frame displayed in the display area 3202 to be streamed or casted to another display device or monitor. When the frame is being streamed or casted to another display device or monitor, a graphical user interface similar to the one shown in FIG. 22 is displayed on the display screen 404 of the imaging device 400.

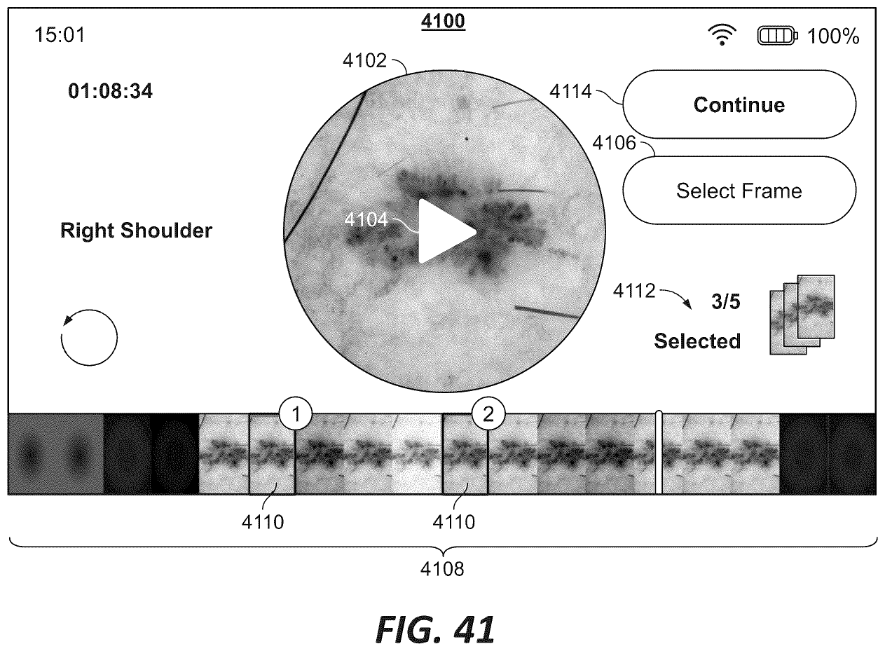

Referring back to FIG. 12, operation 1230 can include displaying a graphical user interface 4100 (FIG. 41) when the type of optical viewing device is a dermatoscope. The graphical user interface 4100 includes a display area 4102 that allows the user to playback the video of the skin area by selecting a playback icon 4104. The graphical user interface 4100 includes a select frame icon 4106 that allows the user to select one or more frames 4110 from a plurality of frames 4108 displayed at the bottom of the graphical user interface 4100. The graphical user interface 4100 can display in an area 4112 the number of frames selected by the user (e.g., 2 frames) out of a maximum number of frames allowed (e.g., 5 frames).

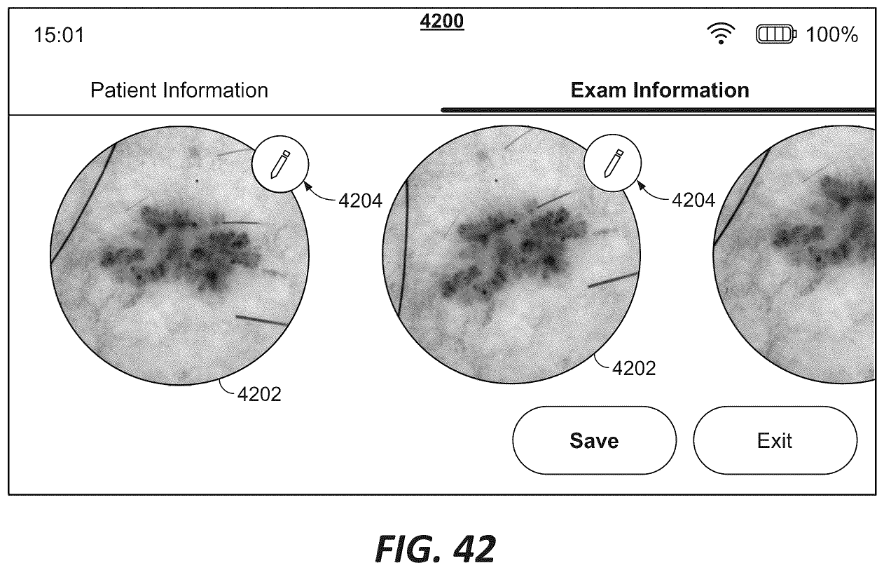

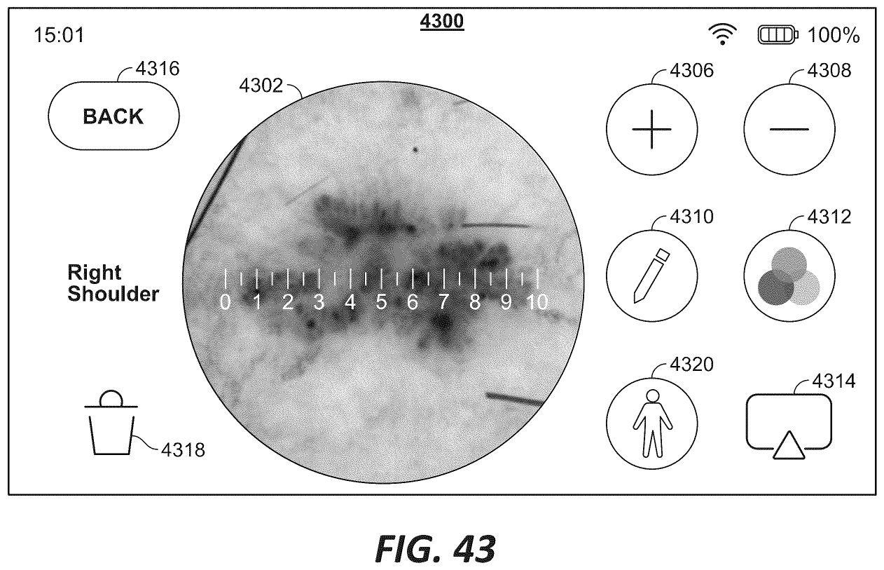

Once the user is satisfied with the selected frames from the video of the left ear or the right ear, the user can select a continue icon 4114. Thereafter, the workflow 1200 can proceed to the operation 1232 allowing the user to customize the one or more frames selected in operation 1230. For example, operation 1232 can include displaying a graphical user interface 4200 (see FIG. 42) that displays one or more frames 4202 selected from the video of the skin area. The user can select an edit icon 4204 which causes a display of a graphical user interface 4300 (see FIG. 43) that allows the user to edit a selected frame of the skin area.

The graphical user interface 4300 includes a display area 4302 of the selected frame of the skin area. The graphical user interface 4300 includes a return icon 4316 that when selected causes the workflow 1200 to return back to the graphical user interface 4200 that displays one or more frames 4202. The graphical user interface 4300 includes a delete icon 4318 that when selected causes the workflow 1200 to delete the selected frame of the skin area.

The graphical user interface 4300 has a zoom-in icon 4306 and a zoom-out icon 4308 that can be selected to zoom in or out the selected frame of the skin area. The graphical user interface 4300 includes an annotations icon 4310 that when selected allows the user to add one or more annotations to the frame displayed in the display area 4302.

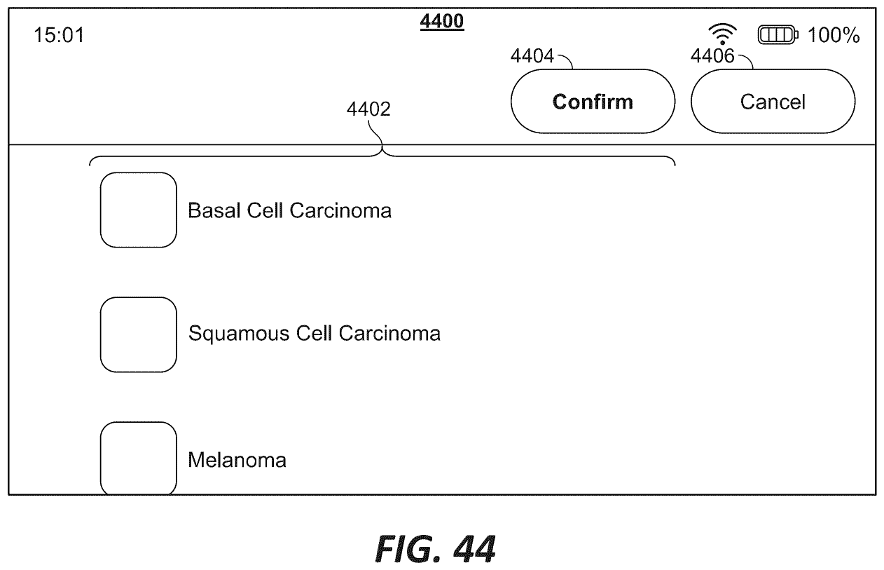

The annotations icon 4310 when selected by the user causes the workflow 1200 to display a graphical user interface 4400 (FIG. 44) that can include one or more predefined annotations 4402 based on the anatomy displayed by the frame. When the imaging device 400 is attached to a dermatoscope such that the anatomy is of a skin area, the one or more predefined annotations 4402 can include selectable options such as basal cell carcinoma, squamous cell carcinoma, or melanoma which may be relevant to a physical assessment of the skin area. The graphical user interface 4400 may also allow the user to type one or more customized annotations. Once the user is satisfied with the annotations, the user can select a confirm icon 4404 to return to the graphical user interface 4300. The user may select a cancel icon 4406 to cancel selections of the one or more annotations and return to the graphical user interface 4300.

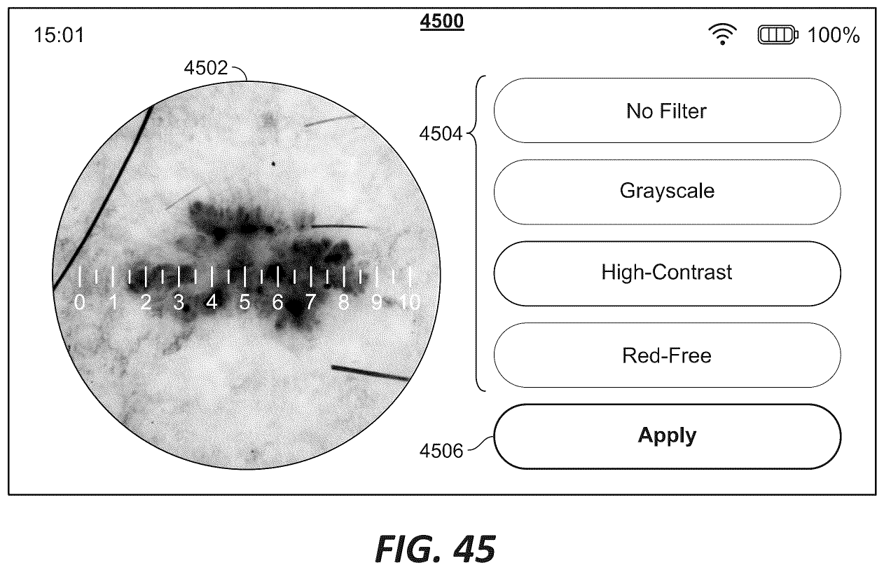

Referring back to FIG. 43, the graphical user interface 4300 can include a filter icon 4312 that when selected allows the user to add one or more filters to the frame displayed in the display area 4302. The filter icon 4312 when selected by the user causes the workflow 1200 to display a graphical user interface 4500 (see FIG. 45) that can include one or more filters 4504 that are displayed based on the anatomy displayed inside a display area 4502. In examples when the imaging device 400 is attached to a dermatoscope such that the anatomy is that of the left or right ear, the one or more filters 4504 can include no filter, grayscale filter, high-contrast filter, and red-free filter. Once the user is satisfied with a selected filter or no filter, the user can select an apply icon 4506 to return to the graphical user interface 4300.

As further shown in FIG. 43, the graphical user interface 4300 can include a casting icon 4314 that causes the frame displayed in the display area 4302 to be streamed or casted to another display device or monitor. When the frame is being streamed or casted to another display device or monitor, a graphical user interface similar to the one shown in FIG. 22 is displayed on the display screen 404 of the imaging device 400. The graphical user interface 4300 can also include an analysis icon 4320 that when selected allows the user to view an analyzed result of the frame displayed in the display area 4302. In some examples, the analyzed result includes an analysis of the frame displayed in the display area 4302 by one or more artificial intelligence algorithms that can screen for one or more types of skin disease.

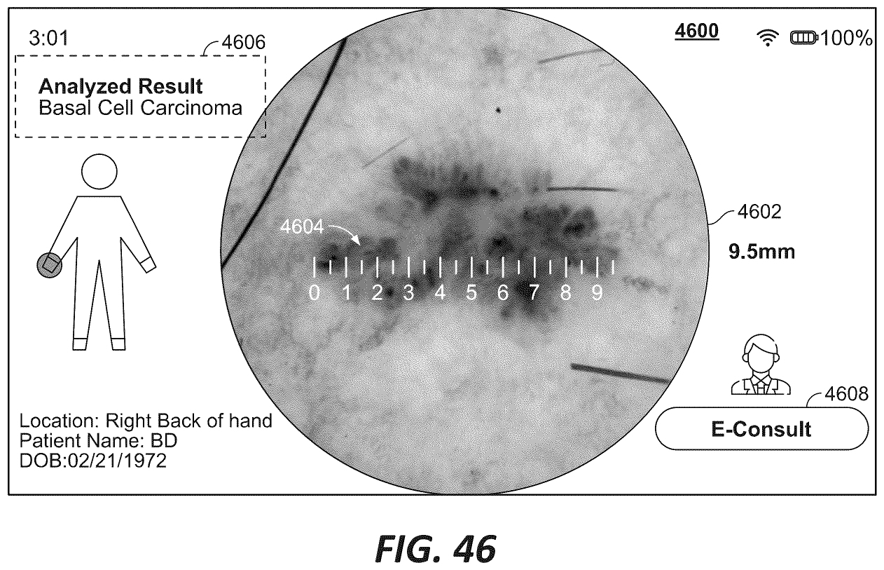

FIG. 46 displays a results interface 4600 that can be displayed on the display screen 404 of the imaging device 400 when the analysis icon 4320 is selected in the graphical user interface 4300 of FIG. 43. The results interface 4600 can display an image of the skin area in a display area 4602. A ruler 4604 can be superimposed over the skin area displayed in the display area 4602. The ruler 4604 can be scaled based on the size of the skin area, or a magnification of the skin area based on the selection of the zoom-in icon 4306 or the zoom-out icon 4308 in the graphical user interface 4300 of FIG. 43. The results interface 4600 further includes a results box 4606 that displays a result from the one or more artificial intelligent algorithms. In the example provided in FIG. 46, the results box 4606 shows a result of basal cell carcinoma. The results interface 4600 can further include a consultation request icon 4608 that when selected causes the workflow to schedule a remote consultation between a specialist (e.g., dermatologist) and a user of the imaging device 400 attached to the third type of optical viewing device 106.

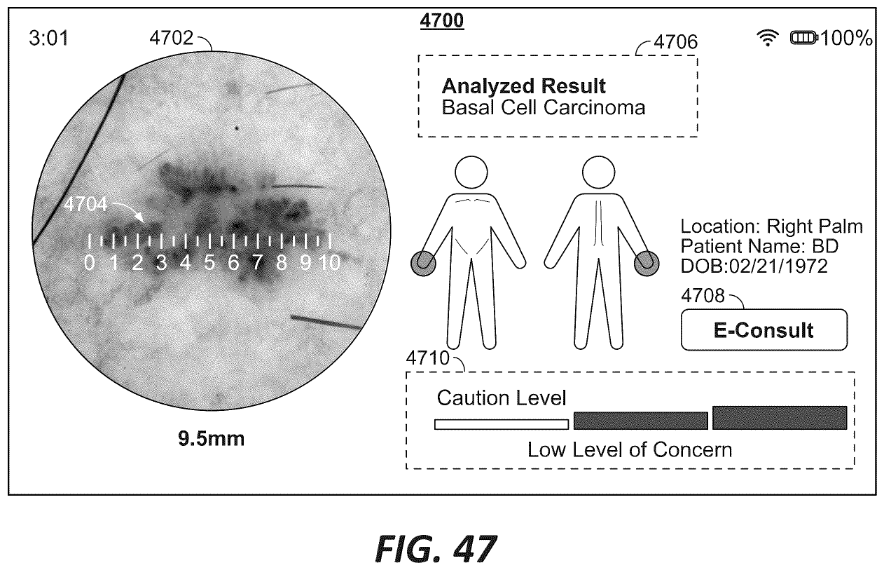

FIG. 47 displays a results interface 4700 that can be displayed on the display screen 404 of the imaging device 400 when the analysis icon 4320 is selected in the graphical user interface 4300 of FIG. 43. The results interface 4700 displays an image of the skin area in a display area 4702, a ruler 4704 is superimposed over the skin area displayed in the display area 4702, a results box 4706 that displays a result from the one or more artificial intelligent algorithms (e.g., basal cell carcinoma), and a consultation request icon 4708 that when selected causes the workflow to schedule a remote consultation between a specialist (e.g., dermatologist) and a user of the imaging device 400. The results interface 4700 can further include a caution level box 4710 that displays a level of caution. In the example shown in FIG. 47, the caution level box 4710 displays a low level of concern. In some examples, the level of concern displayed in the caution level box 4710 is set or adjusted by the user of the imaging device 400. In other examples, the one or more artificial intelligence algorithms set or adjust the level of concern displayed in the caution level box 4710 included in the results interface 4700.

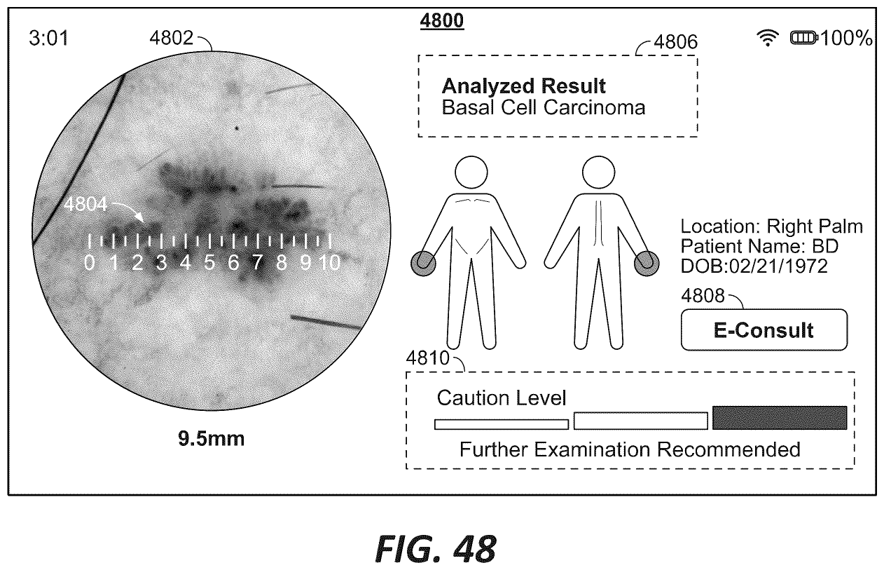

FIG. 48 displays a results interface 4800 that can be displayed on the display screen 404 of the imaging device 400 when the analysis icon 4320 is selected in the graphical user interface 4300 of FIG. 43. The results interface 4800 displays an image of the skin area in a display area 4802, a ruler 4804 is superimposed over the skin area displayed in the display area 4802, a results box 4806 that displays a result from the one or more artificial intelligent algorithms (e.g., basal cell carcinoma), and a consultation request icon 4808 that when selected causes the workflow to schedule a remote consultation between a specialist (e.g., dermatologist) and a user of the imaging device 400. The results interface 4800 further includes a caution level box 4810 that displays a level of caution. In the example shown in FIG. 48, the caution level box 4810 displays a medium level of concern such that further examination is recommended.

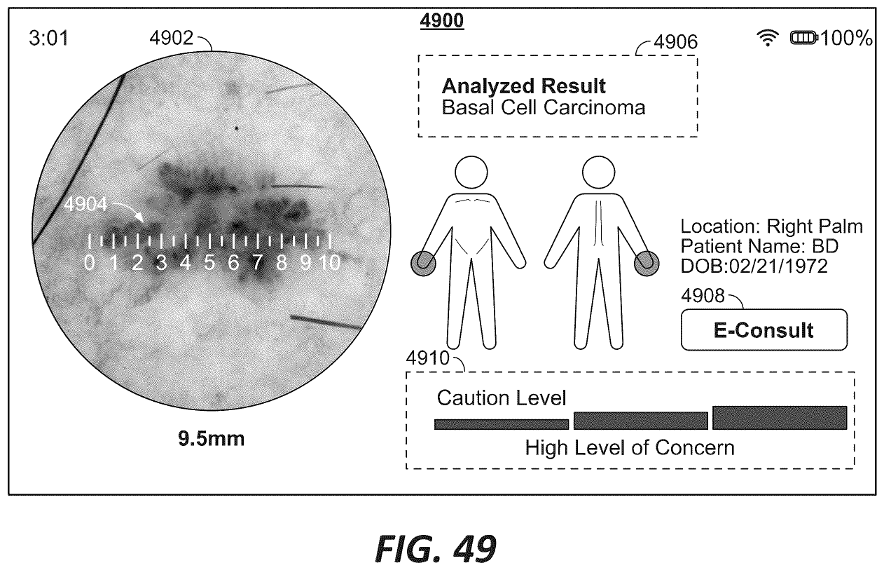

FIG. 49 displays a results interface 4900 that can be displayed on the display screen 404 of the imaging device 400 when the analysis icon 4320 is selected in the graphical user interface 4300 of FIG. 43. The results interface 4900 displays an image of the skin area in a display area 4902, a ruler 4904 is superimposed over the skin area displayed in the display area 4902, a results box 4906 that displays a result from the one or more artificial intelligent algorithms (e.g., basal cell carcinoma), and a consultation request icon 4908 that when selected causes the workflow to schedule a remote consultation between a specialist (e.g., dermatologist) and a user of the imaging device 400. The results interface 4900 further includes a caution level box 4910 that displays a high level of concern.

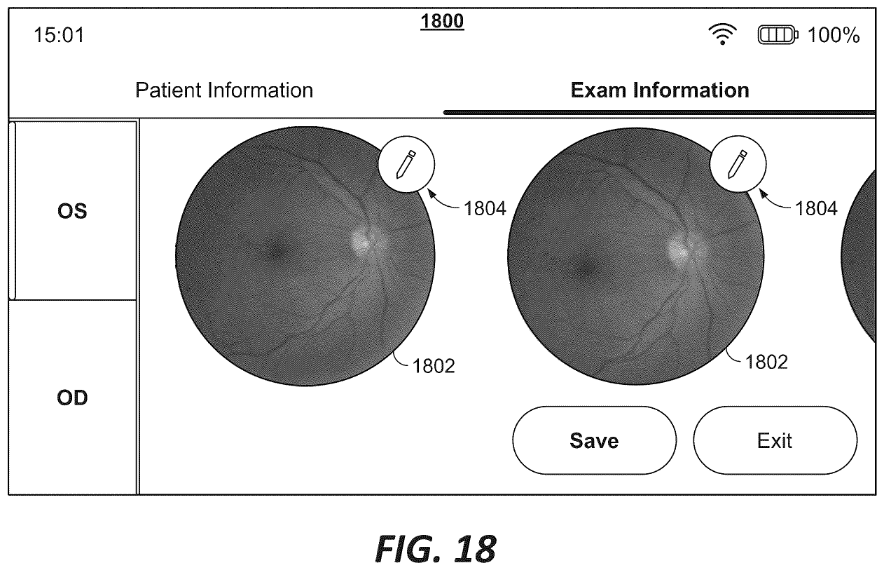

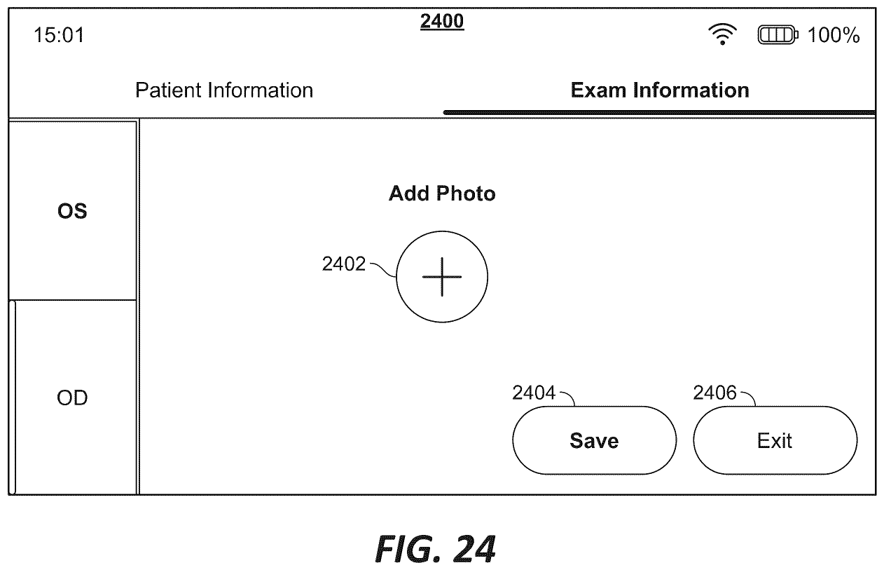

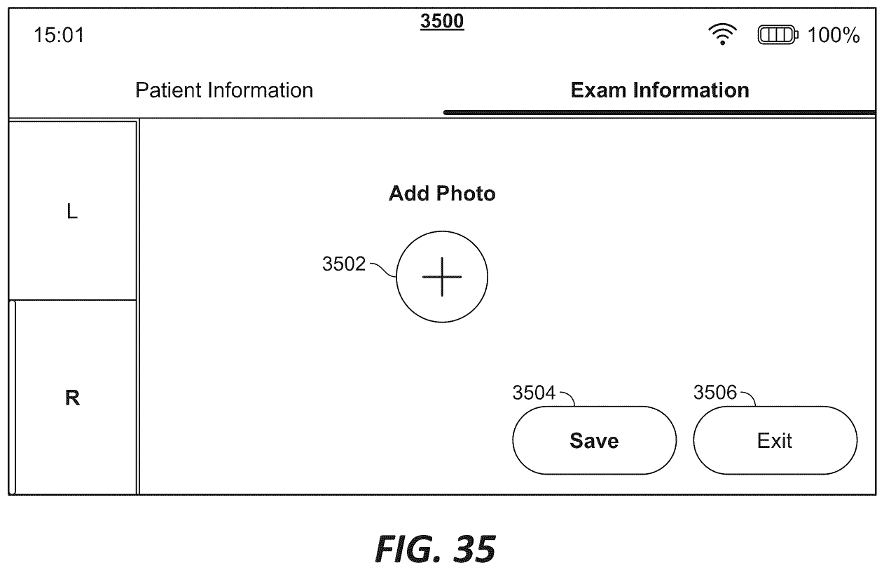

Referring back to FIG. 12, the workflow 1200 can include an operation 1234 of determining whether the user desires to add another video or frame of the anatomy captured by the imaging device 400. The user can scroll through the one or more frames 1802 of the graphical user interface 1800 of FIG. 18 such as by swiping their finger on the display screen 404 to reach an end of the one or more frames (see graphical user interfaces 2400, 3500 shown in FIGS. 24 and 35, respectively). The user can select an add photo icon 2402, 3502 to add another video or frame of the anatomy captured by the camera 410 of the imaging device 400.

In examples where the imaging device 400 is attached to an ophthalmoscope and operation 1234 determines that the user desires to add another video or frame of the left eye (OS) or right eye (OD) (i.e., “Yes” in operation 1234), the imaging device 400 can return to the graphical user interface 1700 of FIG. 17 allowing the user to select another frame 1710 from the plurality of frames 1708 of a video captured by the camera 410 of the left eye (OS) or right eye (OD). In such instances, the workflow 1200 repeats operations 1230-1232. Alternatively, the add photo icon 2402 when selected causes the imaging device 400 to return to the graphical user interface 1300 of FIG. 13 allowing the user to select the same anatomy or a different anatomy to capture another video of the left eye (OS) or the right eye (OD). In such instances, the workflow 1200 repeats operations 1220-1232 for the same eye or the other eye.

In examples where the imaging device 400 is attached to an otoscope and operation 1234 determines that the user desires to add another video or frame of the left ear or right ear (i.e., “Yes” in operation 1234), the imaging device 400 can return to the graphical user interface 3000 of FIG. 30 allowing the user to select another frame 3010 from the plurality of frames 3008 of a video captured by the camera 410 of the left ear or right ear. In such instances, the workflow 1200 repeats operations 1230-1232. Alternatively, the add photo icon 3502 when selected causes the imaging device 400 to return to the graphical user interface 2800 of FIG. 28 allowing the user to select the same anatomy or a different anatomy to capture another video of the left ear or the right ear. In such instances, the workflow 1200 repeats operations 1220-1232.

In examples where the imaging device 400 is attached to a dermatoscope and operation 1234 determines that the user desires to add another video or frame of the skin (i.e., “Yes” in operation 1234), the imaging device 400 can return to the graphical user interface 4200 of FIG. 41 allowing the user to select another frame 4110 from the plurality of frames 4108 of a video captured by the camera 410 of the skin area. In such instances, the workflow 1200 repeats operations 1230-1232. Alternatively, when operation 1234 determines that the user desires to add another video or frame of the skin (i.e., “Yes” in operation 1234), the imaging device 400 returns to the graphical user interface 3700 of FIG. 37 allowing the user to capture another video of the same skin area or a different skin area on the body profile 3702 included in the graphical user interface 3700. In such instances, the workflow 1200 repeats operations 1220-1232.

Referring back to FIG. 12, when operation 1234 determines that the user does not desire to add another video or frame of an anatomy (i.e., “No” in operation 1234), the workflow 1200 can proceed to an operation 1236 of determining whether the user desires to save the video and the annotated frames of the anatomy captured by the camera 410 of the imaging device 400.

As an illustrative example, the workflow 1200 determines the user desires to save the video and the annotated frames of the anatomy (i.e., “Yes” in operation 1236) when the save icon 2404 is selected in the graphical user interface 2400 of FIG. 24. Alternatively, the workflow 1200 determines the user does not desire to save the video and the annotated frames of the anatomy (i.e., “No” in operation 1236) when the exit icon 2406 is selected.

As another illustrative example, the workflow 1200 determines the user desires to save the video and the annotated frames of the anatomy (i.e., “Yes” in operation 1236) when the save icon 3504 is selected in the graphical user interface 3500 of FIG. 35. Alternatively, the workflow 1200 determines the user does not desire to save the video and the annotated frames of the anatomy (i.e., “No” in operation 1236) when the exit icon 3506 is selected. The workflow 1200 can include similar operations when the imaging device 400 is attached to a dermatoscope.

Referring back to FIG. 12, when the workflow 1200 determines the user does not desire to save the video and the annotated frames of the anatomy (i.e., “No” in operation 1236), the video and the annotated frames of the anatomy are deleted from the imaging device 400, and the workflow 1200 ends at operation 1226. Otherwise, when the workflow 1200 determines the user desires to save the video and the annotated frames of the anatomy (i.e., “Yes” in operation 1236), the workflow 1200 includes an operation 1238 of offloading the video and the annotated frames of the anatomy to the external system 600 (see FIG. 1). As described above, in some examples, the external system 600 can host the EMR of the patient such that the video and the annotated frames of the anatomy are stored in the EMR of the patient.

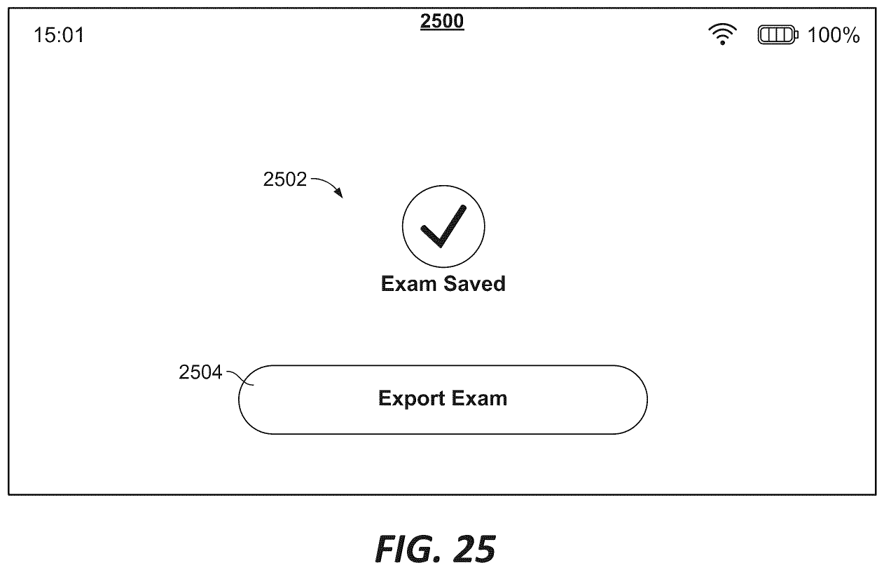

FIG. 25 illustrates an example of a graphical user interface 2500 displayed by the imaging device 400 when the save icon 2404 is selected in the graphical user interface 2400 of FIG. 24. As shown in FIG. 25, the graphical user interface 2500 displays a confirmation 2502 that the video and annotated frames of the anatomy are saved, and includes an export exam icon 2504 that is selectable for exporting the video and annotated frames of the anatomy to the external system 600. Similar graphical user interfaces can be displayed on the imaging device 400 when attached to the otoscope or the dermatoscope.

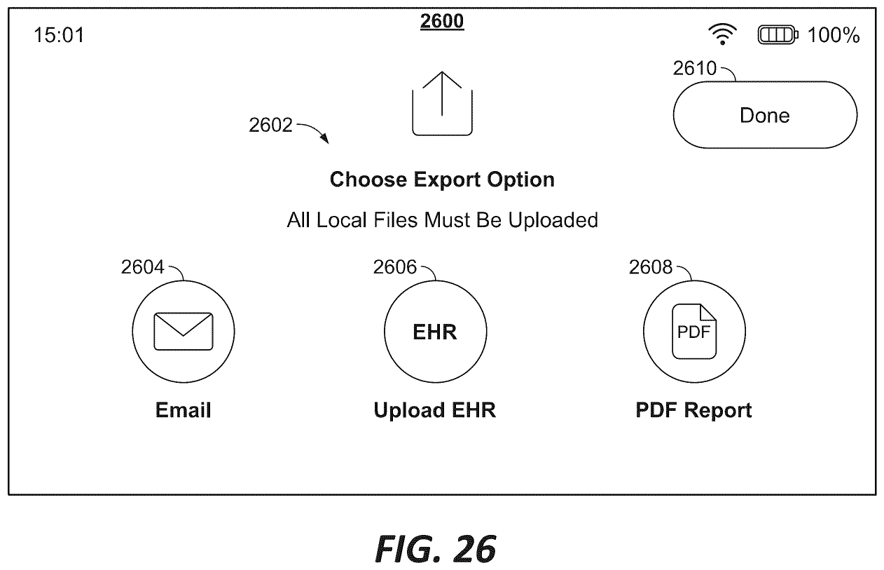

FIG. 26 illustrates an example of a graphical user interface 2600 displayed by the imaging device 400 when the export exam icon 2504 is selected in the graphical user interface 2500 of FIG. 25. As shown in FIG. 26, the graphical user interface 2600 displays a warning 2602 that all local files including the video and annotated frames of the anatomy must be uploaded or discarded from the imaging device 400. This is to ensure protection of patient health information. Similar graphical user interfaces can be displayed on the imaging device 400 when attached to the otoscope or the dermatoscope.

The graphical user interface 2600 includes a first option 2604 to export the video and annotated frames of the anatomy to a trusted email account such as an email of the medical professional who is operating the imaging device attached to the optical viewing device, or to an email of the medical facility where the imaging device attached to the optical viewing device are being used. The graphical user interface 2600 includes a second option 2606 to upload the video and annotated frames of the anatomy to an electronic medical record (EMR) or electronical health record of the patient such as by uploading the video and annotated frames of the anatomy to the external system 600 via the network 5052 (see FIG. 1). The graphical user interface 2600 includes a third option 2608 to generate a report in portable document format (PDF) based on the video and annotated frames of the anatomy which can be exported to an email account or uploaded to the external system 600 via the network 5052 (see FIG. 1).

FIG. 50 illustrates an exemplary architecture of a computing device 5000 of the imaging device 400. The computing device 5000 is used to execute the functionality of the imaging device 400 described herein. The imaging device 400 can include all or some of the elements described with reference to FIG. 50, with or without additional elements.

The computing device 5000 includes at least one processing device 5002. Examples of the at least one processing device 5002 can include central processing units (CPUs), digital signal processors, field-programmable gate arrays, and other types of electronic computing circuits. The at least one processing device 5002 can be part of a processing circuitry having a memory for storing instructions which, when executed by the processing circuitry, cause the processing circuitry to perform the functionalities described herein.

The computing device 5000 also includes a system memory 5004, and a system bus 5006 that couples various system components including the system memory 5004 to the at least one processing device 5002. The system bus 5006 can include any type of bus structure including a memory bus, or memory controller, a peripheral bus, and a local bus.

The system memory 5004 may include a read only memory (ROM) 5008 and a random-access memory (RAM) 5010. An input/output system containing routines to transfer information within the computing device 5000, such as during start up, can be stored in the read only memory (ROM) 5008. The system memory 5004 can be housed inside the housing 402.

The computing device 5000 can further include a secondary storage device 5014 for storing digital data. The secondary storage device 5014 is connected to the system bus 5006 by a secondary storage interface 5016. The secondary storage devices and computer-readable media provide nonvolatile storage of computer-readable instructions including application programs and program devices, data structures, and other data.

A number of program devices can be stored in secondary storage device 5014 or the system memory 5004, including an operating system 5018, one or more application programs 5020, other program devices 5022, and program data 5024. The system memory 5004 and the secondary storage device 5014 are examples of computer-readable data storage devices.

The computing device 5000 can include one or more input devices such as the display screen 404 (in examples where the display screen 404 is a touch sensitive touchscreen), one or more physical push buttons on the housing 402 of the imaging device 400, and the camera 410. Additional examples of input devices include a microphone 5026, and an accelerometer 5028 for image orientation on the display screen 404. The computing device 5000 can also include output devices such as the display screen 404, and a speaker 5030.

The input and output devices are connected to the at least one processing device 5002 through an input/output interface 5038 coupled to the system bus 5006. The input and output devices can be connected by any number of input/output interfaces, such as a parallel port, serial port, game port, or a universal serial bus. Wireless communication between the input and output devices and the input/output interface 5038 is possible as well, and can include Wi-Fi, Bluetooth, infrared, 802.11a/b/g/n, cellular, or other wireless communications.

In some examples, the display screen 404 is touch sensitive and is connected to the system bus 5006 via an interface, such as a video adapter 5042. The display screen 404 includes touch sensors for receiving input from a user when the user touches the display. Such sensors can be capacitive sensors, pressure sensors, or other touch sensors. The sensors detect contact with the display, and also the location and movement of the contact over time. For example, a user can move a finger or stylus across the display screen 404 to provide inputs.

The computing device 5000 further includes a communication device 5046 configured to establish communication across a network 5052. In some examples, when used in a local area networking environment or a wide area networking environment (such as the Internet), the computing device 5000 is typically connected to the network 5052 through a network interface, such as a wireless network interface 5050. The wireless network interface 5050 can provide Wi-Fi functionality such as for image and video transferring, live streaming, and providing a mobile hotspot. In some further examples, the wireless network interface 5050 can provide Bluetooth connectivity. Other possible examples using other wired and/or wireless communications are possible. For example, the computing device 5000 can include an Ethernet network interface, or a modem for communicating across the network.

In further examples, the communication device 5046 provides short-range wireless communication. The short-range wireless communication can include one-way or two-way short-range to medium-range wireless communication. Short-range wireless communication can be established according to various technologies and protocols. Examples of short-range wireless communication include a radio frequency identification (RFID), a near field communication (NFC), a Bluetooth technology, a Wi-Fi technology, or similar wireless technologies.

The computing device 5000 typically includes at least some form of computer-readable media. Computer-readable media includes any available media that can be accessed by the computing device 5000. By way of example, computer-readable media can include computer-readable storage media and computer-readable communication media.

Computer-readable storage media includes volatile and nonvolatile, removable, and non-removable media implemented in any device configured to store information such as computer-readable instructions, data structures, program devices, or other data. Computer-readable storage media includes, but is not limited to, random access memory, read only memory, electrically erasable programmable read only memory, flash memory or other memory technology, or any other medium that can be used to store the desired information and that can be accessed by the computing device 5000.

Computer-readable communication media embodies computer-readable instructions, data structures, program devices or other data in a modulated data signal such as a carrier wave or other transport mechanism and includes any information delivery media. Modulated data signal refers to a signal having one or more of its characteristics set or changed in such a manner as to encode information in the signal. By way of example, computer-readable communication media includes wired media such as a wired network or direct-wired connection, and wireless media such as acoustic, radio frequency, infrared, and other wireless media. Combinations of any of the above are also included within the scope of computer-readable media.

The computing device 5000 is an example of programmable electronics, which may include one or more computing devices, and when multiple computing devices are included, such computing devices can be coupled together with a suitable data communication network so as to collectively perform the various functions, methods, or operations disclosed herein.

The computing device 5000 can include a location identification device 5048. The location identification device 5048 is configured to identify the geolocation or geoposition of the computing device 5000. The location identification device 5048 can use various types of geolocating or positioning systems, such as network-based systems, handset-based systems, SIM-based systems, Wi-Fi positioning systems, and hybrid positioning systems. Network-based systems utilize service provider's network infrastructure, such as cell tower triangulation. Handset-based systems typically use the Global Positioning System (GPS). Wi-Fi positioning systems can be used when GPS is inadequate due to various causes including multipath and signal blockage indoors. Hybrid positioning systems use a combination of network-based and handset-based technologies for location determination, such as Assisted GPS.

The various embodiments described above are provided by way of illustration only and should not be construed to be limiting in any way. Various modifications can be made to the embodiments described above without departing from the true spirit and scope of the disclosure.

What is claimed is:

1. A device for imaging an anatomy, the device comprising:
a bracket for attaching the device to an instrument head of an optical viewing device, the instrument head including an eyepiece for examining the anatomy;
a camera that captures images through the eyepiece of the instrument head;
a display screen that displays the images captured by the camera;
at least one processing device; and
at least one computer readable data storage device storing software instructions that, when executed by the at least one processing device, cause the device to:
determine a type of the optical viewing device; and
present a workflow on the display screen based on the type of the optical viewing device, the workflow enabling capture of a video of the anatomy viewed through the eyepiece of the instrument head, and the workflow including one or more tools for annotating one or more frames selected from the video.

2. The device of claim 1, wherein the type of the optical viewing device is from a group consisting of an ophthalmoscope, an otoscope, and a dermatoscope.

3. The device of claim 1, wherein the instructions, when executed by the at least one processing device, further cause the device to:
determine a user of the imaging device; and
modify the workflow based on the user.

4. The device of claim 1, wherein the instructions, when executed by the at least one processing device, further cause the device to:
determine a patient undergoing an examination by the optical viewing device; and
modify the workflow based on the patient.

5. The device of claim 1, wherein the instructions, when executed by the at least one processing device, further cause the device to:
determine an environment of the optical viewing device; and
modify the workflow based on the environment.

6. The device of claim 1, wherein the one or more tools for annotating the one or more frames selected from the video include at least one of a predefined annotation and a filter.

7. The device of claim 1, wherein the instructions, when executed by the at least one processing device, further cause the device to:
automatically detect the type of the optical viewing device based on imaging performed by the camera of the imaging device, machine-readable data provided by the instrument head, or a connection established between the imaging device and the instrument head.

8. A method of imaging an anatomy, the method comprising:
determining a type of an optical viewing device, the type of the optical viewing device selected from the group consisting of an ophthalmoscope, an otoscope, and a dermatoscope; and
presenting a workflow on the imaging device, the workflow being presented based on the type of the optical viewing device, the workflow enabling capture of a video of the anatomy viewed through an eyepiece of an instrument head, and the workflow including one or more tools for annotating one or more frames selected from the video.

9. The method of claim 8, further comprising:
determining a user of the imaging device; and
modifying the workflow based on the user.

10. The method of claim 8, further comprising:
determining a patient undergoing an examination by the optical viewing device; and
modifying the workflow based on the patient.

11. The method of claim 8, further comprising:
determining an environment of the optical viewing device; and
modifying the workflow based on the environment.

12. The method of claim 8, wherein the one or more tools for annotating the one or more frames selected from the video include one or more predefined annotations based on the anatomy, the one or more predefined annotations being selectable.

13. The method of claim 8, wherein the one or more tools for annotating the one or more frames selected from the video include one or more filters based on the anatomy, the one or more filters being selectable for each frame of the one or more frames.

14. The method of claim 8, further comprising:
capturing the video of the anatomy viewed through the eyepiece of the instrument head.

15. A system for imaging an anatomy, the system comprising:
an optical viewing device including an instrument head having an eyepiece for examining the anatomy; and
an imaging device configured for attachment to the optical viewing device, the imaging device including:
a camera for capturing images through the eyepiece of the instrument head;
a display screen for displaying the images captured by the camera;
at least one processing device communicatively connected to the camera and the display screen; and
at least one computer readable data storage device storing software instructions that, when executed by the at least one processing device, cause the imaging device to:
determine a type of the optical viewing device; and
present a workflow on the display screen based on the type of the optical viewing device, the workflow enabling capture of a video of the anatomy viewed through the eyepiece of the instrument head, and the workflow including one or more tools for annotating one or more frames selected from the video.

16. The system of claim 15, wherein the type of the optical viewing device is from a group consisting of an ophthalmoscope, an otoscope, and a dermatoscope.

17. The system of claim 15, wherein the instructions, when executed by the at least one processing device, further cause the imaging device to:
determine a user of the imaging device; and
modify the workflow based on the user.

18. The system of claim 15, wherein the instructions, when executed by the at least one processing device, further cause the imaging device to:
determine a patient undergoing an examination by the optical viewing device; and
modify the workflow based on the patient.

19. The system of claim 15, wherein the instructions, when executed by the at least one processing device, further cause the imaging device to:
determine an environment of the optical viewing device; and
modify the workflow based on the environment.

20. The system of claim 15, wherein the one or more tools for annotating the one or more frames selected from the video include:
one or more predefined annotations based on the anatomy, the one or more predefined annotations being selectable for each frame of the one or more frames; and
one or more filters based on the anatomy, the one or more filters being selectable for each frame of the one or more frames.
